SEQUENCE LISTING (39 sequences)

SEQ ID NO: 1 - DNA, 840 bp, Homo sapiens
  CDS (76)...(636)
  sig_peptide (76)...(138)
  mat_peptide (139)...(636)
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 2 - protein, 187 aa, Homo sapiens
  SIGNAL (1)...(21)
  CHAIN (22)...(187)
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - DNA, 614 bp, Artificial Sequence
  Synthetic construct
cgtttaaacttaagcttcgccaccatgaccaacaagtgcctgctccagatcgccctgctcctgtgcttcagcaccacggccctatcgatgagctacaacctgctcggcttcctgcagaggagttcgaacttccagtgccagaagctcctgtggcagctgaacgggcgcctggagtactgcctgaaggacaggatgaacttcgacatccccgaggaaatcaagcagctgcagcagttccagaaggaggacgccgctctgaccatctacgagatgctgcagaacatcttcgccatcttccgccaggactccagctccaccggttggaacgagaccatcgtggagaacctgctggccaacgtgtaccaccagatcaaccacctgaagaccgtgctggaggagaagctggagaaggaggacttcacccgcggcaagctgatgagctccctgcacctgaagcgctactatggccgcatcctgcactacctgaaggccaaggagtacagccactgcgcctggaccatcgtacgcgtggagatcctgcgcaacttctacttcatcaaccgcctgaccggctacctgcgcaactgataaggatccactagtccagtgtggtg

SEQ ID NO: 4 - protein, 4 aa, Artificial Sequence
  Synthetic construct
INAX

SEQ ID NO: 5 - protein, 4 aa, Artificial Sequence
  Synthetic construct
GNIX

SEQ ID NO: 6 - protein, 4 aa, Artificial Sequence
  Synthetic construct
VNIX

SEQ ID NO: 7 - protein, 4 aa, Artificial Sequence
  Synthetic construct
SNIX

SEQ ID NO: 8 - protein, 5 aa, Artificial Sequence
  Synthetic construct
ASNIX

SEQ ID NO: 9 - protein, 6 aa, Artificial Sequence
  Synthetic construct
SPINAX

SEQ ID NO: 10 - protein, 7 aa, Artificial Sequence
  Synthetic construct
ASPINAX

SEQ ID NO: 11 - protein, 11 aa, Artificial Sequence
  Synthetic construct
ANIXANIXANI

SEQ ID NO: 12 - protein, 14 aa, Artificial Sequence
  Synthetic construct
ANIXGSNIXGSNIX

SEQ ID NO: 13 - DNA, 40 bp, Artificial Sequence
  Synthetic construct
ccgtcagatcctaggctagcttattgcggtagtttatcac

SEQ ID NO: 14 - DNA, 32 bp, Artificial Sequence
  Synthetic construct
gagctcggtaccaagcttttaagagctgtaat

SEQ ID NO: 15 - DNA, 77 bp, Artificial Sequence
  Synthetic construct
gctgaacgggcgcctggagtactgcctgaaggacaggatgaacttcgacatccccgaggaaatccgccagctgcagc

SEQ ID NO: 16 - DNA, 35 bp, Artificial Sequence
  Synthetic construct
tctccacgcgtacgatggtccaggcgcagtggctg

SEQ ID NO: 17 - DNA, 70 bp, Artificial Sequence
  Synthetic construct
caccacactggactagtggatccttatcagttgcgcaggtagccggtcaggcggttgatgaagtagaagt

SEQ ID NO: 18 - DNA, 31 bp, Artificial Sequence
  Synthetic construct
catcagcttgccggtggtgttgtcctccttc

SEQ ID NO: 19 - DNA, 31 bp, Artificial Sequence
  Synthetic construct
gaaggaggacaacaccaccggcaagctgatg

SEQ ID NO: 20 - DNA, 40 bp, Artificial Sequence
  Synthetic construct
cacactggactagtaagcttttatcagttgcgcaggtagc

SEQ ID NO: 21 - DNA, 47 bp, Artificial Sequence
  Synthetic construct
gaggagttcgaacttccagtgccagcgcctcctgtggcagctgaacg

SEQ ID NO: 22 - DNA, 33 bp, Artificial Sequence
  Synthetic construct
tttaaactggatccagccaccatgaccaacaag

SEQ ID NO: 23 - DNA, 63 bp, Artificial Sequence
  Synthetic construct
cggccatagtagcgcttcaggtgcagggagctcatcagcttgccggtggtgttgtcctccttc

SEQ ID NO: 24 - DNA, 62 bp, Artificial Sequence
  Synthetic construct
gaaggaggacaacaccaccggcaagctgatgagctccctgcacctgaagcgctactatggcc

SEQ ID NO: 25 - DNA, 29 bp, Artificial Sequence
  Synthetic construct
cgcggatccatatgaccaacaagtgcctg

SEQ ID NO: 26 - DNA, 30 bp, Artificial Sequence
  Synthetic construct
ggcgtcctccttggtgaagttctgcagctg

SEQ ID NO: 27 - DNA, 39 bp, Artificial Sequence
  Synthetic construct
atatatcccaagcttttatcagttgcgcaggtagccggt

SEQ ID NO: 28 - DNA, 30 bp, Artificial Sequence
  Synthetic construct
cagctgcagaacttcaccaaggaggacgcc

SEQ ID NO: 29 - DNA, 34 bp, Artificial Sequence
  Synthetic construct
cgcggatccagccaccatgaccaacaagtgcctg

SEQ ID NO: 30 - DNA, 43 bp, Artificial Sequence
  Synthetic construct
caggttgtagctcatattccggagataccccgtcaggcggttg

SEQ ID NO: 31 - DNA, 65 bp, Artificial Sequence
  Synthetic construct
ggtatctccggaatatgagctacaacctgctcggcttcctgcagcgcagttcgaatttccagtgc

SEQ ID NO: 32 - DNA, 18 bp, Artificial Sequence
  Synthetic construct
aagaaggcacagtcgagg

SEQ ID NO: 33 - protein, 15 aa, Artificial Sequence
  Synthetic linker peptide
GSTSGSSGKSSEGKG

SEQ ID NO: 34 - protein, 15 aa, Artificial Sequence
  Synthetic linker peptide
GGGGSGGGGSGGGGS

SEQ ID NO: 35 - protein, 15 aa, Artificial Sequence
  Synthetic linker peptide
GGGGSGGGNSTGGGS

SEQ ID NO: 36 - DNA, 20 bp, Artificial Sequence
  Synthetic primer
ggcacctattggtcttactg

SEQ ID NO: 37 - DNA, 65 bp, Artificial Sequence
  Synthetic primer
gcgggggaggcagcggtggcgggaactccaccggtggcgggagcatgagctacaacctgctcggc

SEQ ID NO: 38 - DNA, 66 bp, Artificial Sequence
  Synthetic primer
gctcccgccaccggtggagttcccgccaccgctgcctcccccgccattccggagataccccgtcag

SEQ ID NO: 39 - DNA, 18 bp, Artificial Sequence
  Synthetic primer
aagaaggcacagtcgagg

(19) United States
(12) Patent Application Publication
(10) Pub. No.: US 2002/0169290 A1
(43) Pub. Date: Nov. 14, 2002

(54) New multimeric interferon beta polypeptides

(76) Inventors: Claus Bornaes, Hellerup (DK); Kim Vilbour Andersen, Broenshoej (DK); Poul Baad Rasmussen, Soeborg (DK); Anders Hjelholt Pedersen, Lyngby (DK)

Correspondence Address: MAXYGEN, INC., 515 GALVESTON DRIVE, RED WOOD CITY, CA, 94063, US

(21) Appl. No.: 10/004,201

(22) Filed: Nov. 1, 2001

Related U.S. Application Data
(60) Provisional application No. 60/245,645, filed on Nov. 2, 2000.

(51) Int. Cl. C07K 14/565

(52) U.S. Cl. 530/351

(57) ABSTRACT

The present invention relates to a single chain multimeric interferon β polypeptide comprising at least two monomers linked via a peptide bond or a peptide linker, wherein at least one of said monomers is an interferon β monomer comprising an amino acid sequence that differs from that of wildtype human interferon β in at least one introduced glycosylation site, methods of preparing such polypeptides or conjugates, and the use of such polypeptides in therapy, in particular for the treatment of multiple sclerosis.

### CROSS-REFERENCE TO RELATED APPLICATIONS

Pursuant to 35 U.S.C. §119(e), this application claims priority to and benefit of U.S. Provisional Patent Application Serial No. 60/245,645 filed on Nov. 2, 2000, the disclosure of which is incorporated herein in its entirety for all purposes.

### COPYRIGHT NOTIFICATION

Pursuant to 37 C.F.R. §1.71(e), Applicants note that a portion of this disclosure contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to single chain multimeric interferon β polypeptides and conjugates thereof, methods of preparing such polypeptides or conjugates, and the use of such polypeptides in therapy, in particular for the treatment of multiple sclerosis.

### BACKGROUND OF THE INVENTION

Interferons are important cytokines characterized by antiviral, antiproliferative, and immunomodulatory activities. These activities form a basis for the clinical benefits that have been observed in a number of diseases, including hepatitis, various cancers and multiple sclerosis. The interferons are divided into the type I and type II classes. Interferon β belongs to the class of type I interferons, which also includes interferons α, τ and ω, whereas interferon γ is the only known member of the distinct type II class.

Human interferon β is a regulatory polypeptide with a molecular weight of 22 kDa consisting of 166 amino acid residues. It can be produced by most cells in the body, in particular fibroblasts, in response to viral infection or exposure to other biologics. It binds to a multimeric cell surface receptor, and productive receptor binding results in a cascade of intracellular events leading to the expression of interferon β inducible genes which in turn produces effects which can be classified as antiviral, antiproliferative and immunomodulatory.

The amino acid sequence of human interferon β was reported by Taniguchi, Gene 10:11-15, 1980, and in EP 83069, EP 41313 and U.S. Pat. No. 4,686,191.

Crystal structures have been reported for human and murine interferon β, respectively (Proc. Natl. Acad. Sci. USA 94:11813-11818, 1997. J. Mol. Biol. 253: 187-207, 1995). They have been reviewed in Cell Mol. Life Sci. 54:1203-1206, 1998.

Relatively few protein-engineered variants of interferon β have been reported (WO 9525170, WO 9848018, U.S. Pat. Nos. 5,545,723, 4,914,033, EP 260350, U.S. Pat. Nos. 4,588,585, 4,769,233, Stewart et al, DNA Vol 6 no2 1987 pp. 119-128, Runkel et al, 1998, Jour. Biol. Chem. 273, No. 14, pp. 8003-8008).

Expression of interferon β in CHO cells has been reported (U.S. Pat. Nos. 4,966,843, 5,376,567 and 5,795,779).

Redlich et al, Proc. Natl. Acad. Sci., USA, Vol. 88, pp. 4040-4044, 1991 disclose immunoreactivity of antibodies against synthetic peptides corresponding to peptide stretches of recombinant human interferon β with the mutation C17S.

Human interferon β molecules with a particular glycosylation pattern and methods for their preparation have been reported (EP 287075 and EP 529300).

Various references disclose modification of polypeptides by polymer conjugation or glycosylation. Polymer modification of native interferon β or a C17S variant thereof has been reported (EP 229108, U.S. Pat. No. 5,382,657, EP 593868, U.S. Pat. No. 4,917,888 and WO 99/55377). U.S. Pat. No. 4,904,584 discloses PEGylated lysine depleted polypeptides, wherein at least one lysine residue has been deleted or replaced with any other amino acid residue. WO 99/67291 discloses a process for conjugating a protein with PEG, wherein at least one amino acid residue on the protein is deleted and the protein is contacted with PEG under conditions sufficient to achieve conjugation to the protein. WO 99/03887 discloses PEGylated variants of polypeptides belonging to the growth hormone superfamily, wherein a cysteine residue has been susbstituted with a non-essential amino acid residue located in a specified region of the polypeptide. Interferon β is mentioned as one example of a polypeptide belonging to the growth hormone superfamily. WO 00/23114 discloses glycosylated and pegylated human interferon β. WO 00/23472 discloses interferon β fusion proteins. WO 00/26354 discloses a method of producing a glycosylated polypeptide variant with reduced allergenicity, which as compared to a corresponding parent polypeptide comprises at least one additional glycosylation site. U.S. Pat. No. 5,218,092 discloses modification of granulocyte colony stimulating factor (G-CSF) and other polypeptides so as to introduce at least one additional carbohydrate chain as compared to the native polypeptide. Interferon β is mentioned as one example among many polypeptides that allegedly can be modified according to the technology described in U.S. Pat. No. 5,218,092.

Commercial preparations of interferon β are sold under the names Betaseron® (also termed interferon β1b, which is non-glycosylated, produced using recombinant bacterial cells, has a deletion of the N-terminal methionine residue and the C17S mutation), and to Avonex™ and Rebif® (also termed interferon β1a, which is glycosylated, produced using recombinant mammalian cells) for treatment of patients with multiple sclerosis, and have shown to be effective in reducing the exacerbation rate, and more patients remain exacerbation-free for prolonged periods of time as compared with placebo-treated patients. Furthermore, the accumulation rate of disability is reduced (Neurol. 51:682-689, 1998).

Comparison of interferon β1a and β1b with respect to structure and function has been presented in Pharmaceut. Res. 15:641-649, 1998.

Interferon β is the first therapeutic intervention shown to delay the progression of multiple sclerosis, a relapsing then progressive inflammatory degenerative disease of the central nervous system. Its mechanism of action, however, remains largely unclear. It appears that interferon β has inhibitory effects on the proliferation of leukocytes and antigen presentation. Furthermore, interferon β may modulate the profile of cytokine production towards an anti-inflammatory phenotype. Finally, interferon β can reduce T-cell migration by inhibiting the activity of T-cell matrix metalloproteases. These activities are likely to act in concert to account for the mechanism of interferon β in MS (Neurol. 51:682-689, 1998).

In addition, interferon β may be used for the treatment of osteosarcoma, basal cell carcinoma, cervical dysplasia, glioma, acute myeloid leukemia, multiple myeloma, Hodgkin's disease, breast carcinoma, melanoma, and viral infections such as papilloma virus, viral hepatitis, herpes genitalis, herpes zoster, herpetic keratitis, herpes simplex, viral encephalitis, cytomegalovirus pneumonia, and rhinovirus.

Various side effects are associated with the use of current preparations of interferon β, including injection site reactions, fever, chills, myalgias, arthralgias, and other flu-like symptoms (Clin. Therapeutics, 19:883-893, 1997).

In addition, 6-40% of patients develop neutralizing antibodies to interferon β (Int. Arch. Allergy Immunol. 118:368-371, 1999). It has been shown that development of interferon β-neutralizing antibodies decreases the biological response to interferon β, and causes a trend towards decreased treatment effect (Neurol. 50:1266-1272, 1998). Neutralizing antibodies will likely also impede the therapeutic utility of interferon β in connection with treatment of other diseases (Immunol. Immuther. 39:263-268, 1994).

Given the magnitude of side effects with current interferon β products, their association with frequent injection, the risk of developing neutralizing antibodies impeding the desired therapeutic effect of interferon β, and the potential for obtaining more optimal therapeutic interferon β levels with concomitant enhanced therapeutic effect, there is clearly a need for improved interferon β-like molecules.

U.S. Pat. No. 5,908,626 discloses hybrid molecules comprising interferon β and an immunoglobulin Fc joined by a peptide linker.

U.S. Pat. No. 4,751,077 discloses modified interferon β molecules, wherein new cysteines are introduced in e.g., position 3 and optionally 101 with the purpose of enabling hybrid formation.

WO 99/38891 discloses modified polypeptides with increased biological activity, in particular modified erythropoietin (EPO), in the form of multimeric polypeptides with polypeptide units covalently linked by thioester bonds.

WO 99/02710 discloses recombinant fusion protein multimers with altered biological activity such as increased plasma half-life, comprising two or more protein molecules fused directly or via a peptide linker. Although this document cites various cytokines, including interferon β, growth factors and hormones, it is in particular directed to modified EPO, and the examples therein relate only to EPO constructs.

It has now been found that it is possible to produce single-chain multimeric interferon β polypeptides and conjugates thereof.

### BRIEF DISCLOSURE OF THE INVENTION

This application discloses single chain multimeric interferon β polypeptides and conjugates contemplated to have a number of improved properties as compared to human interferon β, including increased functional in vivo half-life, increased serum half-life, reduced immunogenicity and/or increased bioavailability. Consequently, the multimeric polypeptide or conjugate of the invention is contemplated to offer a number of advantages over the currently available interferon β compounds, including longer duration between injections, fewer side effects, and/or increased efficiency due to reduction in antibodies. Moreover, higher doses of active protein and thus a more effective therapeutic response may be obtained by use of a conjugate of the invention.

In one aspect the invention relates to a single chain multimeric interferon β polypeptide comprising at least two monomers linked via a peptide bond or a peptide linker, wherein at least one of said monomers is an interferon β monomer that comprises an amino acid sequence which differ from that of wildtype human interferon β in at least one substitution of an amino acid residue of wildtype human interferon β located in a position selected from the group consisting of L5, F8, F15, C17, L47, F50, M62, N80, E81, T82, V101, L106, F111, L116, L120 and F156.

In another aspect the invention relates to a single chain multimeric interferon β polypeptide comprising at least two monomers linked via a peptide bond or a peptide linker, wherein at least one of said monomers is an interferon β monomer that comprises an amino acid sequence which differ from that of wildtype human interferon β in at least one introduced glycosylation site, the glycosylation site preferably being introduced in a position that in wildtype human interferon β is occupied by a surface exposed amino acid residue.

In a still further aspect the invention relates to a single chain multimeric interferon β polypeptide comprising at least two monomers linked via a peptide bond or a peptide linker, wherein at least one of said monomers is an interferon β monomer comprising an amino acid sequence that differs from that of wildtype human interferon β in at least one introduced and/or at least one removed amino acid residue comprising an attachment group for a polymer molecule.

In a still further aspect the invention relates to a single chain multimeric interferon β polypeptide comprising at least two monomers linked via a peptide linker, wherein the peptide linker comprises at least one amino acid residue comprising an attachment group for a non-polypeptide moiety.

In a still further aspect the invention relates to a single chain multimeric interferon β polypeptide comprising at least two monomers linked via a peptide bond or a peptide linker, which further comprises at least one N-terminal or C-terminal peptide addition comprising at least one attachment group for a non-polypeptide moiety.

In a still further aspect the invention relates to a conjugate of a single chain multimeric polypeptide of the invention comprising at least one first non-polypeptide moiety covalently attached to the polypeptide

In a still further aspect the invention relates to a conjugate of a multimeric interferon β polypeptide comprising at least two monomers linked via a peptide bond or a peptide linker, the conjugate further comprising at least one first polymer molecule.

In still further aspects the invention relates to means and methods for preparing a multimeric polypeptide or conjugate of the invention, including nucleotide sequences and expression vectors encoding the polypeptide as well as methods for preparing the polypeptide or the conjugate.

In final aspects the invention relates to a therapeutic composition comprising a multimeric polypeptide or conjugate of the invention, to a multimeric polypeptide, conjugate or composition of the invention for use in therapy, to the use of a multimeric polypeptide, conjugate or composition in therapy or for the manufacture of a medicament for treatment of diseases.

### DETAILED DISCLOSURE OF THE INVENTION

In the present application a number of references are referred to. They are all intended to be incorporated herein by reference.

Definitions

In the context of the present application and invention the following definitions apply:

The term “conjugate” (or interchangeably “conjugated polypeptide”) is intended to indicate a heterogeneous (in the sense of composite or chimeric) molecule formed by the covalent attachment of one or more polypeptide(s) to one or more non-polypeptide moieties. The term covalent attachment means that the polypeptide and the non-polypeptide moiety are either directly covalently joined to one another, or else are indirectly covalently joined to one another through an intervening moiety or moieties, such as a bridge, spacer, or linkage moiety or moieties using an attachment group present in the polypeptide. Preferably, the conjugate is soluble at relevant concentrations and conditions, i.e., soluble in physiological fluids such as blood. Examples of conjugated polypeptides of the invention include glycosylated and/or PEGylated polypeptides. The term “non-conjugated polypeptide” may be used about the polypeptide part of the conjugate.

The term “non-polypeptide moiety” is intended to indicate a molecule that is capable of conjugating to an attachment group of a polypeptide. Preferred examples of such molecule include polymer molecules, sugar moieties, lipophilic compounds, or organic derivatizing agents. When used in the context of a conjugate of the invention it will be understood that the non-polypeptide moiety is linked to the polypeptide part of the conjugate through an attachment group of the polypeptide.

The term “polymer molecule” is defined as a molecule formed by covalent linkage of two or more monomers, wherein none of the monomers is an amino acid residue, except where the polymer is human albumin or another abundant plasma protein. The term “polymer” may be used interchangeably with the term “polymer molecule.” The term is intended to cover carbohydrate molecules attached by in vitro glycosylation, i.e., a synthetic glycosylation performed in vitro normally involving covalently linking a carbohydrate molecule to an attachment group of the polypeptide, optionally using a cross-linking agent. Carbohydrate molecules attached by in vivo glycosylation, such as N- or O-glycosylation (as further described below)) are referred to herein as “a sugar moiety.” Except where the number of non-polypeptide moieties, such as polymer molecule(s) or sugar moieties in the conjugate is expressly indicated every reference to “a non-polypeptide moiety” contained in a conjugate or otherwise used in the present invention shall be a reference to one or more non-polypeptide moieties, such as polymer molecule(s) or sugar moieties, in the conjugate.

The term “attachment group” is intended to indicate an amino acid residue group of the polypeptide capable of coupling to the relevant non-polypeptide moiety. For instance, for polymer, in particular PEG, conjugation a frequently used attachment group is the ε-amino group of lysine or the N-terminal amino group. Other polymer attachment groups include a free carboxylic acid group (e.g., that of the C-terminal amino acid residue or of an aspartic acid or glutamic acid residue), suitably activated carbonyl groups, oxidized carbohydrate moieties and mercapto groups.

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ any amino acid residue that may or may not be identical to X′ and preferably is different from proline, N is asparagine and S/T/C is either serine, threonine or cysteine, preferably serine or threonine, and most preferably threonine). Although the asparagine residue of the N-glycosylation site is the one to which the sugar moiety is attached during glycosylation, such attachment cannot be achieved unless the other amino acid residues of the N-glycosylation site is present. Accordingly, when the non-polypeptide moiety is an N-linked sugar moiety, the term “amino acid residue comprising an attachment group for the non-polypeptide moiety” as used in connection with alterations of the amino acid sequence of the parent polypeptide is to be understood as amino acid residues constituting an N-glycosylation site is/are to be altered in such a manner that either a functional N-glycosylation site is introduced into the amino acid sequence or removed from said sequence. For an “O-glycosylation site” the attachment group is the OH-group of a serine or threonine residue.

The term “one difference” or “differs from” as used in connection with specific mutations is intended to allow for additional differences being present apart from the specified amino acid difference. For instance, in case of removal and/or introduction of amino acid residues comprising an attachment group for the non-polypeptide moiety the interferon β polypeptide may comprise other substitutions that are not related to introduction and/or removal of such amino acid residues. The term “at least one” as used about a non-polypeptide moiety, an amino acid residue, a substitution, etc is intended to mean one or more. The terms “mutation” and “substitution” are used interchangeably herein.

In the present application, amino acid names and atom names (e.g., CA, CB, CD, CG, SG, NZ, N, O, C, etc) are used as defined by the Protein DataBank (PDB) (www.pdb.org) which are based on the IUPAC nomenclature (IUPAC Nomenclature and Symbolism for Amino Acids and Peptides (residue names, atom names e.t.c.), Eur. J. Biochem., 138, 9-37 (1984) together with their corrections in Eur. J. Biochem., 152, 1 (1985). CA is sometimes referred to as Cα, CB as Cβ. The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The terminology used for identifying amino acid positions/substitutions is illustrated as follows: C17 (indicates position #17 occupied by a cysteine residue in the amino acid sequence shown in SEQ ID NO 2). C17S (indicates that the cysteine residue of position 17 has been replaced with a serine). The numbering of amino acid residues made herein is made relative to the amino acid sequence shown in SEQ ID NO 2. “M1del” is used about a deletion of the methionine residue occupying position 1. Multiple substitutions are indicated with a “+,” e.g., R71N+D73T/S means an amino acid sequence which comprises a substitution of the arginine residue in position 71 with an asparagine and a substitution of the aspartic acid residue in position 73 with a threonine or serine residue, preferably a threonine residue. T/S as used about a given substitution herein means either a T or a S residue, preferably a T residue.

The term “nucleotide sequence” is intended to indicate a consecutive stretch of two or more nucleotide molecules. The nucleotide sequence may be of genomic, cDNA, RNA, semisynthetic, synthetic origin, or any combinations thereof.

The term “interferon β protein sequence family” is used in its conventional meaning, i.e., to indicate a group of polypeptides with sufficiently homologous amino acid sequences to allow alignment of the sequences, e.g., using the CLUSTALW program. An interferon β sequence family is available, e.g., from the PFAM families, version 4.0, or may be prepared by use of a suitable computer program such as CLUSTALW CLUSTAL W Multiple Sequence Alignment Program (version 1.8, June 1999) using default parameters (Thompson et al., 1994, CLUSTAL W: improving the sensitivity of progressive multiple sequence alignment through sequence weighting, position-specific gap penalties and weight matrix choice, Nucleic Acids Research, 22:4673-4680).

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

“Cell,” “host cell,” “cell line” and “cell culture” are used interchangeably herein and all such terms should be understood to include progeny resulting from growth or culturing of a cell. “Transformnation” and “transfection” are used interchangeably to refer to the process of introducing DNA into a cell.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

The term “introduce” is primarily intended to mean substitution of an existing amino acid residue, but may also mean insertion of an additional amino acid residue. The term “remove” is primarily intended to mean substitution of the amino acid residue to be removed by another amino acid residue, but may also mean deletion (without substitution) of the amino acid residue to be removed.

The term “immunogenicity” as used in connection with a given substance is intended to indicate the ability of the substance to induce a response from the immune system. The immune response may be a cell or antibody mediated response (see, e.g., Roitt: Essential Immunology (8^{th} Edition, Blackwell) for further definition of immunogenicity). Immunogenicity may be determined by use of any suitable method known in the art, e.g., in vivo or in vitro, e.g., using the in vitro immunogenicity test outlined in the Materials and Methods section below. The term “reduced immunogenicity” is intended to indicate that the conjugate or multimeric polypeptide of the present invention gives rise to a measurably lower immune response than a reference molecule, such as wildtype human interferon β, e.g., Rebif or Avonex, or a variant of wild-type human interferon β such as Betaseron, as determined under comparable conditions. When reference is made herein to commercially available interferon β products (i.e., Betaseron, Avonex and Rebif), it should be understood to mean either the formulated product or the interferon β polypeptide part of the product (as appropriate). Normally, reduced antibody reactivity (e.g., reactivity towards antibodies present in serum from patients treated with commercial interferon β products) is an indication of reduced immunogenicity.

The term “functional in vivo half-life” is used in its normal meaning, i.e., the time at which 50% of a given functionality of the polypeptide or conjugate is retained (such as the time at which 50% of the biological activity of the polypeptide or conjugate is still present in the body/target organ, or the time at which the activity of the polypeptide or conjugate is 50% of the initial value). As an alternative to determining functional in vivo half-life, “serum half-life” may be determined, i.e., the time in which 50% of the polypeptide or conjugate molecules circulate in the plasma or bloodstream prior to being cleared. Determination of serum half-life is often more simple than determining functional in vivo half-life and the magnitude of serum half-life is usually a good indication of the magnitude of functional in vivo half-life. Alternative terms to serum half-life include “plasma half-life,” “circulating half-life,” “serum clearance,” “plasma clearance” and “clearance half-life.” The functionality to be retained is normally selected from antiviral, antiproliferative, immunomodulatory or receptor binding activity. Functional in vivo half-life and serum half-life may be determined by any suitable method known in the art as further discussed in the Materials and Methods section hereinafter.

The polypeptide or conjugate is normally cleared by the action of one or more of the reticuloendothelial systems (RES), kidney, spleen or liver, or by specific or unspecific proteolysis. Clearance taking place by the kidneys may also be referred to as “renal clearance” and is e.g., accomplished by glomerular filtration, tubular excretion or tubular elimination. Normally, clearance depends on physical characteristics of the conjugate, including molecular weight, size (diameter) (relative to the cut-off for glomerular filtration), charge, symmetry, shape/rigidity, attached carbohydrate chains, and the presence of cellular receptors for the protein. A molecular weight of about 67 kDa is considered to be an important cut-off-value for renal clearance.

Reduced renal clearance may be established by any suitable assay, e.g., an established in vivo assay. Typically, the renal clearance is determined by administering a labelled (e.g., radiolabelled or fluorescence labelled) polypeptide conjugate to a patient and measuring the label activity in urine collected from the patient. Reduced renal clearance is determined relative to the corresponding non-conjugated polypeptide or the non-conjugated corresponding wild-type polypeptide or a commercial interferon β product under comparable conditions.

The term “increased” as used about the functional in vivo half-life or serum half-life is used to indicate that the relevant half-life of the conjugate or multimeric polypeptide is statistically significantly increased relative to that of a reference molecule, such as an conjugated wildtype human interferon β (e.g., Avonex or Rebif) or an unconjugated variant human interferon β (e.g., Betaseron) as determined under comparable conditions.

The term “reduced immunogenicity and/or increased functional in vivo half-life and/or increased serum half-life” is to be understood as covering any one, two or all of these properties. Preferably, a conjugate or polypeptide of the invention has at least two or these properties, i.e., reduced immunogenicity and increased functional in vivo half-life, reduced immunogenicity and increased serum half-life or increased functional in vivo half-life and increased serum half-life. Most preferably, the conjugate or polypeptide of the invention has all properties.

The term “exhibiting interferon β activity” is intended to indicate that the polypeptide or conjugate has one or more of the functions of native interferon β, in particular human wildtype interferon β with the amino acid sequence shown in SEQ ID NO 2 (which is the mature sequence) optionally expressed in a glycosylating host cell or any of the commercially available interferon β products. Such functions include capability to bind to an interferon receptor that is capable of binding interferon β and initiating intracellular signalling from the receptor, in particular a type I interferon receptor constituted by the receptor subunits IFNAR-2 and IFNAR-1 (Domanski et al., The Journal of Biological Chemistry, Vol. 273, No. 6, pp3144-3147, 1998, Mogensen et al., Journal of Interferon and Cytokine Research, 19: 1069-1098, 1999), and antiviral, antiproliferative or immunomodulatory activity (which can be determined using assays known in the art (e.g., those cited in the following disclosure)). Interferon β activity may be assayed by methods known in the art as exemplified in the Materials and Methods section hereinafter.

The polypeptide or conjugate “exhibiting” or “having” interferon β activity is considered to have such activity, when it displays a measurable function, e.g., a measurable receptor binding and stimulating activity (e.g., as determined by the primary or secondary assay described in the Materials and Methods section). The polypeptide exhibiting interferon β activity may also be termed “interferon β molecule” or “interferon β polypeptide” herein. When in monomeric form, the interferon β polypeptide may be termed “interferon β monomer”; when in multimeric form “interferon β multimer” or “multimeric interferon β polypeptide.”

The term “parent interferon β” is normally used about a starting interferon β monomer molecule to be modified as described herein. While the parent interferon β may be of any origin, such as vertebrate or mammalian origin (e.g., any of the origins defined in WO 00/23472), the parent interferon β is preferably wild-type human interferon β with SEQ ID NO 2 or a variant thereof. A “variant” is a polypeptide, which differs in one or more amino acid residues from a parent polypeptide, normally in 1, 2, 3, 4, 5, 6, 7, 8, 9, 10, 11, 12, 13, 14 or 15 amino acid residues, and which exhibits interferon β activity. Examples of wild-type human interferon β include the polypeptide part of Avonex or Rebif. An example of a parent interferon β variant is Betaseron. Alternatively, the parent interferon β polypeptide may comprise an amino acid sequence, which is a hybrid molecule between interferon β and another homologous polypeptide, such as interferon α, optionally containing one or more additional substitutions introduced into the hybrid molecule. Such a hybrid molecule may contain an amino acid sequence, which differs in more than 10 amino acid residues from the amino acid sequence shown in SEQ ID NO 2. In order to be useful in the present invention the hybrid molecule exhibits interferon β activity (e.g., as determined in the secondary assay described in the Materials and Methods section herein).

The term “functional site” as used about a polypeptide or conjugate of the invention is intended to indicate one or more amino acid residues which is/are essential for or otherwise involved in the function or performance of interferon β, and thus “located at” the functional site. The functional site is e.g., a receptor binding site and may be determined by methods known in the art, preferably by analysis of a structure of the polypeptide complexed to a relevant receptor, such as the type I interferon receptor constituted by IFNAR-1 and IFNAR-2.

Single Chain Multimeric Polypeptide of the Invention

General Considerations

The multimeric interferon β polypeptide of the invention comprises two or more monomers, normally two or three monomers. However, also multimeric polypeptides comprising more than three monomers are contemplated, such as four, five, six or more monomers.

It will be understood that the individual monomers to be used in the construction of the multimeric polypeptide are selected so as to provide it with the desired activity or property, that, e.g., includes increased half-life, reduced or no immunogenicity, increased bioavailability, etc., in addition to interferon β activity. For this purpose at least one of the monomers should exhibit interferon β activity. Also, it is desirable that all of the monomer constituents are low or non-immunogenic in the patient for which the multimeric product is intended. The latter may, e.g., be achieved either by using human wildtype interferon β or a low or non-immunogenic variant thereof, e.g., any of the variants disclosed in the present application.

In one embodiment, all monomers of the multimeric polypeptide of the invention or for use in preparing a conjugate of the invention are interferon β monomers, preferably human wildtype interferon β or a variant thereof as disclosed herein. However, the invention is not limited to this since it is presently believed that multimeric interferon β polypeptides comprising only one interferon β monomer and one or more other monomers exhibiting significantly reduced or no interferon β activity can be constructed. The one or more other monomers are normally non- or low immunogenic in the patient to be treated, comprise at least 80 such as at least 100 amino acid residues, and furthermore exhibit an amino acid sequence identity to human wildtype interferon β of at least 80% with the amino acid sequence of wildtype human interferon β, in particular at least 90% such as at least 95% identity as determined by use of Clustal W. Thus, the multimeric interferon β polypeptide of the invention may comprise one interferon β monomer and one or more other monomers, two interferon β monomers and one or more other monomers, etc.

The individual monomers may have identical or different amino acid sequences. For instance, a dimeric interferon β polypeptide according to the invention may comprise e.g., two identical or two different interferon β monomers linked by a peptide bond or a peptide linker. Preferably, the interferon β monomers are selected from the group consisting of human wildtype interferon β and a variant thereof, e.g., any of those described herein. For instance the dimer may comprise one interferon β monomer truncated by one or two C-terminal amino acid residues linked, optionally via a peptide linker, to a second interferon β monomer truncated in one or two N-terminal residues, The same principles for combining different interferon β monomers are of course also valid for other multimers such as trimers, tetramers, pentamers, etc. In the case of a trimer, for example, it may comprise three identical monomers (e.g., wildtype human interferon β or a variant thereof, e.g., as disclosed herein), two identical monomers (e.g., wild-type human interferon β or a variant thereof, e.g., as disclosed herein) and one monomer that is different from the two (e.g., a variant), or three different monomers.

Furthermore, when the interferon β monomer is wildtype human interferon β or a variant thereof it may be provided in truncated form, i.e., truncated in one or more N-terminal or C-terminal amino acid residues as compared to the parent interferon β molecule. For instance, the interferon β monomer may be truncated in one or two N-terminal amino acid residues and/or in one or two C-terminal amino acid residues. The truncation is preferably done in the region of the monomer intended to be linked to a second monomer or a peptide linker. This may serve to increase the proteolytic stability of the resulting multimeric interferon β polypeptide.

The monomers used for constructing the multimeric polypeptide may be linked by a peptide bond, or may be connected by a suitable linker peptide. If used, the linker peptide must be of a type (length, amino acid composition, amino acid sequence, etc) that is adequate to link two (or more) monomers in such a way that they assume a conformation relative to one another so that the resulting multimeric polypeptide has interferon β activity. Furthermore, the linker peptide is typically designed to increase the stability of the resulting multimeric polypeptide towards proteolytic degradation, e.g by use of special amino acid sequences or residues. When the multimeric polypeptide is intended for conjugation to a non-polypeptide moiety, the peptide linker sequence may comprise one or more attachment groups for said non-polypeptide moiety. For instance, when the non-polypeptide moiety is a sugar moiety the linker can contain the sequence NAT providing an N-glycosylation site. Alternatively, when a the non-polypeptide moiety is a sugar moiety the linker can contain the sequence NST providing an N-glycosylation site. In one embodiment the linker has one N-glycosylation site. When the non-polypeptide moiety is a PEG, the linker can contain, e.g., Lys or Cys.

For instance, the linker peptide predominantly includes the amino acid residues Gly, Ser, Ala or Thr. The linker may also include Lys, Asn, or Glu. The linker typically comprises 1-30 amino acid residues, such as a sequence of about 5-30, 2-20, 3-15, or 10-20 amino acid residues, or 15 amino acid residues. Likewise, the amino acid residues selected for inclusion in the linker peptide should exhibit properties that do not interfere significantly with the interferon β activity of the multimeric polypeptide. Thus, the linker peptide should on the whole not exhibit a charge which would be inconsistent with the interferon β activity of the multimeric polypeptide, or interfere with internal folding, or form bonds or other interactions with amino acid residues in one or more of the monomers which would seriously impede the binding of the multimeric polypeptide to the ligand-binding domain of the receptor.

Specific linkers for use in the present invention may be designed on the basis of known naturally occurring as well as artificial polypeptide linkers (see, e.g., Hallewell et al. (1989), J. Biol. Chem. 264, 5260-5268; Alfthan et al. (1995), Protein Eng. 8, 725-731; Robinson & Sauer (1996), Biochemistry 35, 109-116; Khandekar et al. (1997), J. Biol. Chem. 272, 32190-32197; Fares et al. (1998), Endocrinology 139, 2459-2464; Smallshaw et al. (1999), Protein Eng. 12, 623-630; U.S. Pat. No. 5,856,456). For instance, linkers used for creating single-chain antibodies, e.g., a 15mer consisting of three repeats of a Gly-Gly-Gly-Gly-Ser amino acid sequence ((Gly₄Ser)₃), are contemplated to be useful in the present invention. Other linkers that contemplated to be useful in the present invention are GlySerThrSerGlySerSerGlyLysSerSer GluGlyLysGly, and GlyGlyGlyGlySerGlyGlyGlyAsnSerThrGlyGlyGlySer. Furthermore, phage display technology as well as selective infective phage technology can be used to diversify and select appropriate linker sequences (Tang et al., J. Biol. Chem. 271, 15682-15686, 1996; Hennecke et al. (1998), Protein Eng. 11, 405-410). Also, the Arc repressor phage display has been used to optimise the linker length and composition for increased stability of the single-chain protein (Robinson and Sauer (1998), Proc. Natl. Acad. Sci. USA 95, 5929-5934).

Another way of obtaining a suitable linker is by optimizing a simple linker—e.g., ((Gly₄Ser)ₙ)—through random mutagenesis.

It will be clear from the present specification that whatever the nature of the linker, it should be one which is not readily susceptible to cleavage by e.g., proteases or chemical agents, since cleavage of the multimeric polypeptide to result in two or more monomeric units is not desired in the present context.

Specific Single Chain Multimeric Polypeptides of the Invention

In the present section specific types of single chain multimeric polypeptides of the invention are described. It will be understood that the general considerations given above apply for each of the multimeric polypeptides described in the present section. Thus, for instance the number and nature of monomer constituents, the type of linker, etc is as described above, unless otherwise indicated.

In a first aspect the invention relates to a single chain multimeric interferon β polypeptide comprising at least two monomers linked via a peptide bond or a peptide linker, wherein at least one of said monomers comprises an amino acid sequence that differs from that of wildtype human interferon β in at least one substitution of an amino acid residue of wildtype human interferon β located in a position selected from the group consisting of L5, F8, F15, C17, L47, F50, M62, N80, E81, T82, V101, L106, F111, L116, L120 and F156. Specific examples of such substitutions C17S, M62A,L,V, N80C, E81C, T82C and V101S. The other monomer(s) are preferably interferon β monomer(s), e.g., selected from human wildtype interferon β or a variant thereof as described herein. The individual substitutions are described previously as giving rise to improved interferon β monomers and are contemplated to be of interest for the production of multimeric polypeptides of the invention. In one embodiment of the invention it relates to a single chain dimer interferon beta polypeptide.

In a second aspect the invention relates to a single chain multimeric interferon β polypeptide comprising at least two monomers linked via a peptide bond or a peptide linker, wherein at least one of said monomers is an interferon β monomer comprising an amino acid sequence that differs from that of wild type human interferon β in at least one introduced glycosylation site, in particular a glycosylation site which has been introduced into a position that in wildtype human interferon β is occupied by a surface exposed amino acid residue. Preferably, the interferon β monomer is selected from those described in the section entitled “Conjugate of the invention wherein the non-polypeptide moiety is a sugar moiety” having at least one introduced glycosylation site. For instance, the multimeric interferon β polypeptide comprises one or two such monomers optionally combined with wildtype human interferon β, or an interferon β monomer as described in connection with the third aspect of the invention. In one embodiment of the multimeric interferon β polypeptide, the interferon β monomer comprises at least one substitution selected from the group consisting of S2N+N4T, L9N+R11T, R11N, S12N+N14T, F15N+C16S, Q16N+Q18T, K19N+L21T, Q23N+H25T, G26N+L28T, R27N+E29T, L28N+Y30T, D39T, K45N+L47T, Q46N+Q48T, Q48N+F50T, Q49N+Q51T, Q51N+E53T, R71N+D73T, Q72N, D73N, S75N, S76N+G78T, L88T, Y92T, N93N+I95T, L98T, E103N+K105T, E104N+L106T, E107N+E109T, K108N+D110T, D110N, F111N+R113T and L116N. In another embodiment of the invention it relates to a single chain dimer interferon beta polypeptide.

In a third aspect the invention relates to a single chain multimeric interferon β polypeptide comprising at least two monomers linked via a peptide bond or a peptide linker, wherein at least one of said monomers is an interferon β monomer that comprises an amino acid sequence that differs from that of wildtype human interferon β in at least one introduced and/or at least one removed amino acid residue comprising an attachment group for a polymer molecule. Preferably, the interferon β monomer is any of those described in the section entitled “Conjugate of the invention wherein the non-polypeptide moiety has a lysine residue as an attachment group,” “Conjugate of the invention wherein the non-polypeptide moiety has a cysteine residue as an attachment group,” and “Conjugate of the invention wherein the non-polypeptide moiety has an acid group as an attachment group.” For instance, the multimeric interferon β polypeptide comprises one or two such monomers optionally combined with wildtype human interferon β, or an interferon β monomer as described in connection with the second aspect of the invention. In an embodiment of the multimeric polypeptide, the at least one introduced amino acid residue is introduced into a position of wildtype human interferon β that is occupied by a surface exposed amino acid residue and/or wherein the at least one removed amino acid residue is surface exposed in wildtype human interferon β. In another embodiment of the multimeric polypeptide, the introduced and/or removed amino acid residue is selected from the group consisting of lysine, cysteine, aspartic acid or glutamic acid. In a further embodiment of the multimeric polypeptide, at least one of the lysine residues K19, K33, K45, K52 and K123 has been removed from at least one of the monomers. In a further embodiment of the multimeric polypeptide, the interferon β monomer comprises at least one substitution selected from the group consisting of N4K, R11K, G26K, R27K, Q48K, Q49K, R71K, D73K, S75K, E85K, A89K, Y92K, H93K, F111K, R113K, L116K, R124K, G127K and Y155K. In a further embodiment of the invention it relates to a single chain dimer interferon beta polypeptide.

In a fourth aspect the invention relates to a single chain multimeric interferon β polypeptide comprising at least two monomers linked via a peptide linker, wherein at least one of the monomers is an interferon β monomer and wherein the peptide linker comprises at least one amino acid residue comprising an attachment group for a non-polypeptide moiety. This aspect is of particular relevance when the multimeric polypeptide is intended to be conjugated to the non-polypeptide moiety, and thereby, for instance, be in the form of a glycosylated or PEGylated multimeric polypeptide. This attachment group (or groups), and the non-polypeptide moieties that may be attached to it/them, may be any of those described elsewhere herein. One or more of the monomers may contain amino acid residues comprising an attachment group for the same non-polypeptide moiety, which may have been introduced therein as described herein. Also, amino acid residues comprising attachment groups for the non-polypeptide moiety may have been removed from one or more of the monomers. For instance, the attachment group may be a glycosylation site, in particular an in vivo glycosylation site, such as an O- or N-glycosylation site. In an embodiment of the multimeric polypeptide, it comprises two or more monomers with the same amino acid sequence. In another embodiment of the multimeric polypeptide it comprises 2 or 3 interferon β monomers. In a further embodiment of the multimeric polypeptide at least one of the monomers is wildtype human interferon β, optionally C-terminally or N-terminally truncated. In a further embodiment of the invention it relates to a single chain dimer interferon beta polypeptide.

In a further aspect the invention relates to a single chain multimeric interferon β polypeptide comprising at least two monomers, of which at least one is an interferon β monomer, linked via a peptide bond or a peptide linker, which further comprises at least one N-terminal or C-terminal peptide addition comprising at least one attachment group for a non-polypeptide moiety. The multimeric interferon β polypeptide is e.g., any of those described in the present application, including a dimer, trimer or higher multimer of human wildtype interferon β. It will be understood that the single chain multimeric interferon β according to this aspect is of particular interest for conjugation to a non-polypeptide moiety. In a further embodiment of the invention it relates to a single chain dimer interferon beta polypeptide.

The term “peptide addition” is intended to indicate one or more consecutive amino acid residues that are added to the C-terminal or N-terminal amino acid of the amino acid sequence of the multimeric polypeptide. Normally, the peptide addition is linked to the amino acid sequence by a peptide linkage.

In principle the peptide addition can be any stretch of amino acid residues ranging from a single amino acid residue to a mature protein. Usually, the peptide addition comprises 1-500 amino acid residues, such as 2-500, normally 2-50 or 3-50 amino acid residues, such as 3-20 amino acid residues. The length of the peptide addition to be used is dependent of or determined on the basis of a number of factors including the specific sequence of the multimeric polypeptide and the desired effect to be achieved by the modification. The peptide addition may be designed by a site-specific or random approach, e.g., as out-lined in further detail in the Methods section below. This section also comprises a set of guidelines useful for preparing a peptide addition for use in the present invention are described. It will be understood that those guidelines are intended for illustration purposes only and that a person skilled in the art will be aware of alternative useful routes for design of peptide addition. Thus, the method of designing a peptide addition for use herein should not be considered limited to that described in the Materials section.

The number of attachment groups should be sufficient to provide the desired effect. Typically, the peptide addition comprises 1-20, such as 1-10 attachment groups for the non-polypeptide moiety. For instance, the peptide addition comprises 1, 2, 3, 4, 5, 6, 7, 8, 9 or 10 attachment groups for the non-polypeptide moiety. It is well known that one frequently occurring consequence of modifying an amino acid sequence of, e.g., a human protein is that new epitopes are created by such modification. In order to shield any new epitopes created by the peptide addition, it is desirable that sufficient attachment groups are present to enable shielding of all epitopes introduced into the sequence. This is e.g., achieved when the peptide addition comprises at least one attachment group within a stretch of 30 contiguous amino acid residues, such as at least one attachment group within 20 amino acid residues or at least one attachment group within 10 amino acid residues, in particular 1-3 attachment groups within a stretch of 10 contiguous amino acid residues in the peptide addition.

Thus, in one embodiment the peptide addition comprises at least two attachment groups for the non-polypeptide moiety, wherein two of said amino acid residues are separated by at most 10 amino acid residues, none of which comprises an attachment group for the non-polypeptide moiety.

Furthermore, the multimeric polypeptide of the invention can comprise at least one introduced attachment group for the non-polypeptide moiety, in particular 1-5 introduced attachment groups. Analogously, the multimeric polypeptide can comprise at least one removed attachment group for the non-polypeptide moiety, in particular 1-5 removed attachment groups.

When the non-polypeptide moiety is an N-linked sugar moiety, examples of suitable peptide additions include INAT/S, GNIT/S, VNIT/S, SNIT/S, ASNIT/S, NIT/S, SPINAT/S, ASPINAT/S, ANIT/SANIT/SANI, and ANIT/SGSNIT/SGSNIT/S, wherein T/S is either a T or an S residue, preferably a T residue. The peptide addition can comprise one or more of these peptide sequences, i.e., at least two of said sequences either directly linked together or separated by one or more amino acid residues, or can contain two or more identical copies of any of these peptide sequence. It will be understood that the above specific sequences are given for illustrative purposes and thus do not constitute an exclusive list of peptide sequences of use in the present invention.

In a more specific embodiment the peptide addition is selected from the group consisting of INAT/S, GNIT/S, VNIT/S, SNIT/S, ASNIT/S, NIT/S, SPINAT/S, ASPINAT/S, ANIT/SANIT/SANI, and ANIT/SGSNIT/SGSNIT/S, wherein T/S is either a T or an S residue, preferably a T residue.

Conjugate of the Invention

In a further aspect the invention relates to a conjugate of a single chain multimeric interferon β polypeptide of the invention (as described above) comprising at least one first non-polypeptide moiety covalently attached to the multimeric polypeptide. The first non-polypeptide moiety may be of any suitable type, as described in further detail in the section below entitled “Non-polypeptide moiety of the conjugate of the invention,” but is preferably a polymer molecule or a sugar moiety.

In a still further aspect the invention relates to a conjugate of a multimeric interferon β polypeptide comprising at least two monomers linked via a peptide bond or a peptide linker, the conjugate further comprising at least one first polymer molecule. In an embodiment of the conjugate at least one of the monomers is an interferon β monomer. In accordance with this aspect the interferon β monomer(s) may be wildtype human interferon β optionally in N-terminally or C-terminally truncated form. For instance, when the multimeric polypeptide is a dimer, both monomers may be wildtype human interferon β optionally in N-terminally or C-terminally truncated form; one monomer may be wildtype human interferon β and one a variant thereof, e.g., any of the variants described in the present application; or both monomers may be variants of wildtype human interferon β.

For instance, the interferon β monomer comprises an amino acid sequence that differs from that of wildtype human interferon β in at least one introduced and/or at least one removed amino acid residue comprising an attachment group for said first non-polypeptide moiety.

In a further embodiment of the conjugate the first non-polypeptide moiety is a polymer molecule, such as 1 to 3 polymer molecules. In another embodiment the first non-polypeptide moiety is a sugar moiety, such as I to 7 sugar moieties.

In a further embodiment of the conjugate the polymer molecule is linear or branched polyethylene glycol. In an embodiment the polymer molecule has lysine or cysteine as an attachment group.

In a further embodiment of the conjugate it further comprises at least one second non-polypeptide moiety. In an embodiment the first non-polypeptide moiety is a polymer molecule and the second non-polypeptide moiety is a sugar moiety, or the first non-polypeptide moiety is a sugar moiety and the second non-polypeptide moiety is a polymer molecule.

In a further embodiment of the conjugate the interferon β monomer comprises an amino acid sequence that differs from that of wild-type human interferon β in that at least one glycosylation site has been introduced or removed and at least one amino acid residue comprising an attachment group for a polymer molecule has been introduced and/or removed.

In a further embodiment of the conjugate it comprises two or more monomers of wild-type human interferon β.

In a further embodiment of the conjugate it comprises at least one wildtype human interferon β monomer and at least one interferon β monomers comprising an amino acid sequence that differs from that of wildtype human interferon β.

In a further embodiment of the conjugate it comprises at least two interferon β monomers that comprises an amino acid sequence that differs from wildtype human interferon β.

In a further embodiment of the conjugate it has at least one improved property selected from the group consisting of reduced immunogenicity, increased functional in vivo half-life and increased serum half-life as compared to Avonex, Rebif or Betaseron.

By removing and/or introducing amino acid residues comprising an attachment group for the non-polypeptide moiety it is possible to specifically adapt the monomer(s) so as to make the molecule more susceptible to conjugation to the non-polypeptide moiety of choice, to optimize the conjugation pattern (e.g., to ensure an optimal distribution of non-polypeptide moieties on the surface of the interferon β molecule and thereby, e.g., effectively shield epitopes and other surface parts of the polypeptide without significantly impairing the function thereof). For instance, by introduction of attachment groups in one or more monomers, the resulting multimeric interferon β polypeptide is boosted or otherwise altered in the content of the specific amino acid residues to which the relevant non-polypeptide moiety binds, whereby a more efficient, specific and/or extensive conjugation is achieved. By removal of one or more attachment groups it is possible to avoid conjugation to the non-polypeptide moiety in parts of the polypeptide in which such conjugation is disadvantageous, e.g., to an amino acid residue located at or near a functional site of the polypeptide (since conjugation at such a site may result in inactivation or reduced interferon β activity of the resulting conjugate due to impaired receptor recognition). Further, it may be advantageous to remove an attachment group located closely to another attachment group in order to avoid heterogeneous conjugation to such groups.

It will be understood that the amino acid residue comprising an attachment group for a non-polypeptide moiety, either it be removed or introduced, is selected on the basis of the nature of the non-polypeptide moiety and, in most instances, on the basis of the conjugation method to be used. For instance, when the non-polypeptide moiety is a polymer molecule, such as a polyethylene glycol or polyalkylene oxide derived molecule, amino acid residues capable of functioning as an attachment group may be selected from the group consisting of lysine, cysteine, aspartic acid, glutamic acid and arginine. When the non-polypeptide moiety is a sugar moiety the attachment group is an in vivo glycosylation site, preferably an N-glycosylation site.

Whenever an attachment group for a non-polypeptide moiety is to be introduced into or removed from the interferon β monomer in accordance with the present invention, the position of the interferon β monomer to be modified is conveniently selected as follows:

The position is preferably located at the surface of the interferon β monomer, and more preferably occupied by an amino acid residue that has more than 25% of its side chain exposed to the solvent, preferably more than 50% of its side chain exposed to the solvent. Such positions have been identified on the basis of an analysis of a 3D structure of the human interferon β molecule as described in the Methods section herein. Also, an attachment group is preferably introduced in the part of the monomer which is to linked to a second monomer or peptide linker of the multimeric interferon β polypeptide of the invention.

Alternatively or additionally, the position to be modified is identified on the basis of an analysis of an interferon β protein sequence family. More specifically, the position to be modified can be one, which in one or more members of the family other than the parent interferon β, is occupied by an amino acid residue comprising the relevant attachment group (when such amino acid residue is to be introduced) or which in the parent interferon β, but not in one or more other members of the family, is occupied by an amino acid residue comprising the relevant attachment group (when such amino acid residue is to be removed).

In order to determine an optimal distribution of attachment groups, the distance between amino acid residues located at the surface of the interferon β monomer is calculated on the basis of a 3D structure thereof. More specifically, the distance between the CB's of the amino acid residues comprising such attachment groups, or the distance between the functional group (NZ for lysine, CG for aspartic acid, CD for glutamic acid, SG for cysteine) of one and the CB of another amino acid residue comprising an attachment group are determined. In case of glycine, CA is used instead of CB. In the multimeric interferon β polypeptide part of a conjugate of the invention, any of said distances is preferably more than 8 Å, in particular more than 10 Å in order to avoid or reduce heterogeneous conjugation.

Furthermore, in the multimeric interferon β polypeptide part of a conjugate of the invention attachment groups located at the receptor-binding site of interferon β has preferably been removed, preferably by substitution of the amino acid residue comprising such group.

A still further generally applicable approach for modifying an interferon β monomer is to shield, and thereby destroy or otherwise inactivate an epitope present in the parent interferon β, by conjugation to a non-polypeptide moiety. Epitopes of human interferon β may be identified by use of methods known in the art, also known as epitope mapping, see, e.g., Romagnoli et al., J. Biol Chem, 1999, 380(5):553-9, DeLisser H M, Methods Mol Biol, 1999, 96:11-20, Van de Water et al., Clin Immunol Immunopathol, 1997, 85(3):229-35, Saint-Remy J M, Toxicology, 1997, 119(1):77-81, and Lane D P and Stephen C W, Curr Opin Immunol, 1993, 5(2):268-71. One method is to establish a phage display library expressing random oligopeptides of e.g., 9 amino acid residues. IgG1 antibodies from specific antisera towards human interferon β are purified by immunoprecipitation and the reactive phages are identified by immunoblotting. By sequencing the DNA of the purified reactive phages, the sequence of the oligopeptide can be determined followed by localization of the sequence on the 3D-structure of the interferon β. Alternatively, epitopes can be identified according to the method described in U.S. Pat. No. 5,041,376. The thereby identified region on the structure constitutes an epitope that then can be selected as a target region for introduction of an attachment group for the non-polypeptide moiety. Preferably, at least one epitope, such as two, three or four epitopes of human recombinant interferon β (optionally comprising the C17S mutation) are shielded by a non-polypeptide moiety according to the present invention. Accordingly, in one embodiment, the conjugate of the invention has at least one shielded epitope as compared to wild type human interferon β, optionally comprising the C17S mutation, including any commercially available interferon β. Preferably, the conjugate of the invention comprises a polypeptide that is modified so as to shield the epitope located in the vicinity of amino acid residue Q49 and/or F111. This may be done by introduction of an attachment group for a non-polypeptide moiety into a position located in the vicinity of (i.e., within 4 amino acid residues in the primary sequence or within about 10 Å in the tertiary sequence) of Q49 and/or F111. The 10 Å distance is measured between CB's (CA's in case of glycine). Such specific introductions are described in the following sections.

In case of removal of an attachment group, the relevant amino acid residue comprising such group and occupying a position as defined above is preferably substituted with a different amino acid residue that does not comprise an attachment group for the non-polypeptide moiety in question.

In case of introduction of an attachment group, an amino acid residue comprising such group is introduced into the position, preferably by substitution of the amino acid residue occupying such position.

The exact number of attachment groups available for conjugation and present in the multimeric interferon β polypeptide is dependent on the effect desired to be achieved by conjugation. The effect to be obtained is, e.g., dependent on the nature and degree of conjugation (e.g., the identity of the non-polypeptide moiety, the number of non-polypeptide moieties desirable or possible to conjugate to the polypeptide, where they should be conjugated or where conjugation should be avoided, etc.). For instance, if reduced immunogenicity is desired, the number (and location of) attachment groups should be sufficient to shield most or all epitopes. This is normally obtained when a greater proportion of the multimeric interferon β polypeptide is shielded. Effective shielding of epitopes is normally achieved when the total number of attachment groups available for conjugation is in the range of 1-10 attachment groups, in particular in the range of 1-7, such as 1-5.

Functional in vivo half-life is i.a. dependent on the molecular weight of the conjugate and the number of attachment groups needed for providing increased half-life thus depends on the molecular weight of the non-polypeptide moiety in question. In one embodiment, the conjugate of the invention has a molecular weight of at least 67 kDa, in particular at least 70 kDa as measured by SDS-PAGE according to Laemmli, U.K., Nature Vol 227 (1970), p680-85. Human wildtype interferon β has a molecular weight of about 20 kDa, and therefore, when the multimeric polypeptide is a dimer comprising two human wildtype interferon β monomers, additional about 30 kDa is required to obtain the desired effect. This may be, e.g., be provided by three 10 kDa PEG molecules or as otherwise described herein.

In order to preserve structure and function of the interferon β monomer constituent(s) of the multimeric interferon β polypeptide of the invention, the total number of amino acid residues to be altered in each monomer (as compared to the amino acid sequence shown in SEQ ID NO 2) typically does not exceed 15. Preferably, the interferon β monomer comprises an amino acid sequence, which differs in 1-15 amino acid residues from the amino acid sequence shown in SEQ ID NO 2, such as in 1-8 or in 2-8 amino acid residues, e.g., in 1-5 or in 2-5 amino acid residues from the amino acid sequence shown in SEQ ID NO 2. Thus, normally the interferon β monomer comprises an amino acid sequence that differs from the amino acid sequence shown in SEQ ID NO 2 in 1, 2, 3, 4, 5, 6, 7, 8, 9, 10, 11, 12, 13, 14 or 15 amino acid residues. Preferably, the above numbers represent either the total number of introduced or the total number of removed amino acid residues comprising an attachment group for the relevant non-polypeptide moiety, or the total number of introduced and removed amino acid residues comprising such group.

In the conjugate of the invention it is preferred that at least about 50% of all conjugatable attachment groups, such as at least about 80% and preferably all of such groups are occupied by the relevant non-polypeptide moiety. Accordingly, in a preferred embodiment the conjugate of the invention comprises, e.g., 1-10 non-polypeptide moieties, such as 2-8 or 3-6.

The conjugate or multimeric polypeptide of the invention has one or more of the following improved properties:

Reduced immunogenicity as compared to wild-type human interferon β (e.g., Avonex or Rebif) or to Betaseron, e.g., a reduction of at least 25%, such as at least 50%, and more preferably at least 75%;

Increased functional in vivo half-life and/or increased serum half-life as compared to wild-type human interferon β (e.g., Avonex or Rebif) or to Betaseron;

Reduced or no reaction with neutralizing antibodies from patients treated with wildtype human interferon β (e.g., Rebif or Avonex) or with Betaseron, e.g., a reduction of neutralisation of at least 25%, such as of at least 50%, and preferably of at least 75%.

The magnitude of the antiviral activity of a conjugate of the invention may not be critical, and thus be reduced (e.g., by up to 75%) or increased (e.g., by at least 5%) or equal to that of wild-type human interferon β ((e.g., Avonex or Rebif) or to Betaseron;

Furthermore, the degree of antiviral activity as compared to antiproliferative activity of a conjugate of the invention may vary, and thus be higher, lower or equal to that of wildtype human interferon β.

Conjugate of the Invention, Wherein the Non-Polypeptide Moiety is a Molecule that Has Lysine as an Attachment Group

The multimeric polypeptide or conjugate of the invention may comprise at least one interferon β monomer that comprises an amino acid sequence that differs from that of wild-type human interferon β in at least one introduced and/or at least one removed lysine residue. The conjugate also comprises at least one first non-polypeptide moiety attached to a lysine residue of the multimeric polypeptide. While the non-polypeptide moiety may be any of those binding to a lysine residue, e.g., the ε-amino group thereof, such as a polymer molecule, a lipophilic group, an organic derivatizing agent or a carbohydrate moiety, it is preferably any of the polymer molecule mentioned in the section entitled “Conjugation to a polymer molecule,” in particular a branched or linear PEG or polyalkylene oxide. Most preferably, the polymer molecule is PEG and the activated molecule to be used for conjugation is SS-PEG, NPC-PEG, aldehyd-PEG, mPEG-SPA, mPEG-SCM, mPEG-BTC from Shearwater Polymers, Inc, SC-PEG from Enzon, Inc., tresylated mPEG as described in U.S. Pat. No. 5,880,255, or oxycarbonyl-oxy-N-dicarboxyimide-PEG (U.S. Pat. No. 5,122,614). Normally, for conjugation to a lysine residue the non-polypeptide moiety has a molecular weight of about 5, 10, 12, or 20 kDa.

In one embodiment the amino acid sequence of the interferon β monomer differs from that of human wildtype interferon β in at least one removed lysine residue, such as 1-5 removed lysine residues, in particular 1-4 or 1-3 removed lysine residues. The lysine residue(s) to be removed, preferably by replacement, is selected from the group consisting of K19,K33,K45,K52,K99,K105,K108,K115,K123,K134, and K136. The lysine residues may be replaced with any other amino acid residue, but is preferably replaced by an arginine or a glutamine residue in order to give rise to the least structural difference. In particular, the polypeptide part may be one, wherein K19, K45, K52 and/or K123, preferably K19, K45 and/or K123 has/have been replaced with another any other amino acid residue, preferably arginine or glutamine. For instance, the interferon β monomer comprises a combination of amino acid substitutions selected from the following list:

K19R+K45R+K123R;

K19Q+K45R+K123R;

K19R+K45Q+K123R;

K19R+K45R+K123Q;

K19Q+K45Q+K123R;

K19R+K45Q+K123Q;

K19Q+K45R+K123Q;

K19Q+K45Q+K123Q;

K19R+K33R+K45R;

K45R+K123R;

K45Q+K123R;

K45Q+K123Q;

K45R+K123Q;

K19R+K123R;

K19Q+K123R;

K19R+K123Q;

K19Q+K123Q;

K19R+K45R;

K19Q+K45R;

K19R+K45Q; or

K19Q+K45Q.

In addition or alternatively to the amino acid substitutions mentioned in the above list the interferon β monomer may comprise at least one substitution selected from the group consisting of K33R, K33Q, K52R, K52Q, K99R, K99Q, K105R, K105Q, K108R, K108Q, K115R, K115Q, K134R, K134Q, K136R, and K136Q, e.g., at least one of the following substitutions:

K52R+K134R;

K99R+K136R;

K33R+K105R+K136R;

K52R+K108R+K134R;

K99R+K115R+K136R;

K19R+K33R+K45R+K123R;

K19R+K45R+K52R+K123R;

K19R+K33R+K45R+K52R+K123R; or

K19R+K45R+K52R+K99R+K123R.

In a further embodiment the amino acid sequence of the interferon β monomer differs from that shown in SEQ ID NO 2 in that a lysine residue has been introduced by substitution of at least one amino acid residue occupying a position that in the parent interferon β molecule is occupied by a surface exposed amino acid residue, preferably an amino acid residue having at least 25%, such as at least 50% of its side chain exposed to the surface. Preferably, the amino acid residue to be substituted is selected from the group consisting of N4, F8, L9, R11, S12, F15, Q16, Q18, L20, W22, Q23, G26, R27, L28, E29, Y30, L32, R35, M36, N37, D39, P41, E42, E43, L47, Q48, Q49, T58, Q64, N65, F67, A68, R71, Q72, D73, S75, S76, G78, N80, E81, I83, E85, N86, A89, N90, Y92, H93, H97, T100, L102, E103, L106, E107, E109, D110, F111, R113, G114, L116, M117, L120, H121, R124, G127, R128, L130, H131, E137, Y138, H140, I145, R147, V148, E149, R152, Y155, F156, N158, R159, G162, Y163, R165 and N166 of SEQ ID NO 2.

More preferably, the amino acid sequence of the interferon β monomer differs from the amino acid sequence shown in SEQ ID NO 2 in that a lysine residue has been introduced, by substitution, of at least one amino acid residue occupying a position selected from the group consisting of N4, F8, L9, R11, S 12, G26, R27, E29, R35, N37, D39, E42, L47, Q48, Q49, A68, R71, Q72, D73, S75, G78, N80, E85, N86, A89, Y92, H93, D110, F111, R113, L116, H121, R124, G127, R128, R147, V148, Y155, N158, R159, G162 and R165, even more preferably selected from the group consisting of N4, R11, G26, R27, Q48, Q49, R71, D73, S75, N80, E85, A89, Y92, H93, F111, R113, L116, R124, G127, R128, Y155, N158 and G162, and most preferably selected from the group consisting of R11, Q49, R71, S75, N80, E85, A89, H93, F111, R113, L116 and Y155, and most preferably Q49 and F111.

In accordance with this embodiment, the interferon β monomer comprises a substitution to lysine in one or more of the above positions, in particular in 1-15, such as 1-8 or 1-5, and preferably in at least two positions, such as 2-8 or 2-5 positions.

In a further embodiment the amino acid sequence of the interferon β monomer differs in at least one removed and at least one introduced lysine residue, such as 1-5 or 2-5 removed lysine residues and 1-5 or 2-5 introduced lysine residues. It will be understood that the lysine residues to be removed and introduced preferably are selected from those described in the present section.

In accordance with this embodiment of the invention, the total number of conjugatable lysine residues in each monomer or in the multimeric polypeptide may be in the range of 1-10, such as 2-8 or 3-7.

For instance, the interferon β monomer may comprise at least one of the following substitutions: R11K, Q48K, Q49K, R71K, S75K, N80K, E85K, A89K, H93K, F111 K, R113K, L116K and Y155K; more preferably R11K, Q49K, R71K, S75K, N80K, E85K, A89K, H93K, F111K, R113K, L116K and Y155K, in combination with at least one of the substitutions: K19R/Q K33R/Q K45R/Q, K52R/Q, K99R/Q, K105R/Q, K108R/Q, K115R/Q, K123R/Q, K134R/Q, and K136R/Q, wherein R/Q indicates substitution to an R or a Q residue, preferably an R residue. More preferably, the interferon β monomer comprises at least one of the following substitutions R11K, Q49K, R71K, S75K, N80K, E85K, A89K, H93K, F111K, R113K, L116K and Y155K, in particular Q49K, F111K and/or N80K, in combination with substitution of at least one of K19, K45, K52 and/or K123, preferably to an R or a Q residue. In particular, the interferon β monomer comprises at least one of the substitutions Q49K, F111K and N80K in combination with at least one of the substitutions mentioned above for removal of a lysine residue. For instance, the interferon β polypeptide monomer may comprise the following substitutions:

Y+Z+K19R+K45R+K123R;

Y+Z+K19Q+K45R+K123R;

Y+Z+K19R+K45Q+K123R;

Y+Z+K19R+K45R+K123Q;

Y+Z+K19Q+K45Q+K123R;

Y+Z+K19R+K45Q+K123Q;

Y+Z+K19Q+K45R+K123Q;

Y+Z+K19Q+K45Q+K123Q;

Y+Z+K45R+K123R;

Y+Z+K45Q+K123R;

Y+Z+K45Q+K123Q;

Y+Z+K45R+K123Q;

Y+Z+K19R+K123R;

Y+Z+K19Q+K123R;

Y+Z+K19R+K123Q;

Y+Z+K19Q+K123Q;

Y+Z+K19R+K45R;

Y+Z+K19Q+K45R;

Y+Z+K19R+K45Q; or

Y+Z+K19Q+K45Q, wherein Y is selected from the group of Q49K, F111K, N80K, Q49K+F111K, Q49K+N80K, F111K+N80K and Q49K+F111K+N80K and Z is absent or comprises at least one substitution selected from the group consisting of K33R, K33Q, K52R, K52Q, K99R, K99Q, K105R, K105Q, K108R, K108Q, K115R, K115Q, K134R, K134Q, K136R, and K136Q. Preferably, the interferon β monomer comprises the following substitution Y+Z+K19R+K45Q+K123R, wherein Y and Z have the above meaning.

More specifically, according to this embodiment the interferon β monomer may comprise one of the following substitutions:

K19R+K45R+F111K+K123R;

K19R+K45R+Q49K+F111K+K123R;

K19R+K45R+Q49K+K123R;

K19R+K45R+F111K;

K19R+K45R+Q49K+F111K;

K19R+Q49K+K123R;

K19R+Q49K+F111K+K123R;

K45Q+F111K+K123Q;

K45R+Q49K+K123R; or

K45R+Q49K+F111K+K123R.

Especially for expression in a non-glycosylating host such as E. coli the interferon β monomer may contain the substitution N80K or C17S+N80K, optionally in combination with one or more of K19R/Q; K45R/Q; K52R/Q or K123R/Q. The substitution N80K is of particular interest, when the single chain multimeric interferon β polypeptide is expressed in a non-glycosylating host cell, since N80 constitutes part of an inherent glycosylation site of human interferon β and conjugation at such site may mimick natural glycosylation.

Furthermore, it is preferred that the conjugate according to this aspect comprises at least two first non-polypeptide moieties, such as 2-8 moieties.

Conjugate of the Invention Wherein the Non-Polypeptide Moiety Binds to a Cysteine Residue

In yet another embodiment the invention relates to a conjugate exhibiting interferon β activity and comprising at least one first non-polypeptide moiety conjugated to at least one cysteine residue of a multimeric interferon β polypeptide, which multimeric polypeptide comprises at least one interferon β monomer comprising an amino acid sequence which differs from that of wild-type human interferon β in at least one introduced cysteine residue in a position that is occupied by a surface exposed amino acid residue.

In a still further embodiment, the invention relates a conjugate exhibiting interferon β activity and comprising at least one first non-polypeptide conjugated to at least one cysteine residue of a single chain multimeric interferon β polypeptide that comprises at least one interferon β monomer that comprising an amino acid sequence which differs from that of wildtype human interferon β in that at least one cysteine residue has been introduced, preferably by substitution, into a position that in the parent interferon β molecule is occupied by an amino acid residue that is exposed to the surface of the molecule, preferably one that has at least 25%, such as at least 50% of its side chain exposed to the surface. For instance, the amino acid residue is selected from the group consisting of F8, L9, R11, S12, F15, Q16, Q18, L20, W22, L28, L32, M36, P41, T58, Q64, N65, F67, I83, E85, N86, A89, N90, Y92, H93, H97, T100, L102, E103, L106, M117, L120, H121, R124, G127, R128, L130, H131, H140, I145, R147, V148, E149, R152, Y155, and F156 of SEQ ID NO 2.

Additionally or alternatively, the substitution is preferably performed at a position occupied by a threonine or serine residue. For instance, such position is selected from the group consisting of S2, S12, S13, T58, S74, S75, S76, T77, T82, T100, T112, S118, S119, S139, T144, and T161, more preferably S2, S12, S13, S74, S75, S76, T77, T82, T100, T112, S118, S119, S139, and T144 (side chain surface exposed), still more preferably S2, S12, S75, S76, T82, T100, S119 and S139 (at least 25% of its side chain exposed), and even more preferably S12, S75, T82 and T100 (at least 50% of its side chain exposed).

Of the above threonine or serine substitutions, serine substitutions are preferred. Accordingly, in even more preferred embodiments, the position is selected from the group consisting of S2, S12, S13, S74, S75, S76, S118, S119 and S139, more preferably S2, S12, S13, S74, S75, S76, S118, S119 and S139, even more preferably S2, S12, S75, S76, S119 and S139, and still more preferably S12 and S75.

In one embodiment, only one cysteine residue is introduced into the interferon β monomer in order to avoid formation of disulphide bridges between two or more introduced cysteine residues. In this connection C17 present in wildtype human interferon β may be removed, preferably by substitution, in particular by substitution with S or A. In another embodiment, two or more cysteine residues are introduced, such as 2-6 or 2-4 cysteine residues. Preferably, the interferon β monomer according to this embodiment of the invention comprises the mutation L47C, Q48C, Q49C, D110C, F111C or R113C, in particular only one of these mutations, optionally in combination with the mutation C17S. Also, the interferon β monomer may comprise the substitution C17S+N80C.

While the first non-polypeptide moiety according to this embodiment of the invention may be any molecule which, when using the given conjugation method has cysteine as an attachment group (such as a carbohydrate moiety, a lipophilic group or an organic derivatizing agent), it is preferred that the non-polypeptide moiety is a polymer molecule. The polymer molecule may be any of the molecules mentioned in the section entitled “Conjugation to a polymer molecule,” but is preferably selected from the group consisting of linear or branched polyethylene glycol or polyalkylene oxide. Most preferably, the polymer molecule is VS-PEG. The conjugation between the polypeptide and the polymer may be achieved in any suitable manner, e.g., as described in the section entitled “Conjugation to a polymer molecule,” e.g., in using a one step method or in the stepwise manner referred to in said section. When the multimeric interferon β polypeptide comprises only one conjugatable cysteine residue, this is preferably conjugated to a first non-polypeptide moiety with a molecular weight of at least 20 kDa, either directly conjugated or indirectly through a low molecular weight polymer (as disclosed in WO 99/55377). When the conjugate comprises two or more first non-polypeptide moieties, normally each of these has a molecular weight of 5 or 10 kDa.

Conjugate of the Invention Wherein the Non-Polypeptide Moiety Has an Acid Group as an Attachment Group

In yet another embodiment the conjugate exhibiting interferon β activity comprises at least one first non-polypeptide moiety having an acid group as an attachment group, which moiety is conjugated to at least one aspartic acid or glutamic acid residue of a multimeric interferon β polypeptide comprising at least one interferon β monomer that comprises an amino acid sequence that differs from that of wild-type human interferon β in at least one introduced and/or at least one removed aspartic acid or glutamic acid residue. The relevant amino acid residue may be introduced in any position of the parent interferon β molecule that is occupied by a surface exposed amino acid residue, preferably by an amino acid residue having more than 25% of its side chain surface exposed. Preferably, at least one amino acid residue occupying a position selected from the group consisting of N4, L5, L6, F8, L9, Q10, R11, S12,S13, F15, Q16, Q18, K19, L20, W22, Q23, L24, N25, G26, R27, Y30, M36, Q46, Q48, Q49, I66, F67, A68, I69, F70, R71, S75, T82, I83, L87, A89, N90, V91, Y92, H93, Q94, I95, N96, H97, K108, F111, L116, L120, K123, R124, Y126, G127, R128, L130, H131, Y132, K134, A135, H140, T144, R147, Y155, F156, N158, R159, G162, Y163 and R165 has been substituted with an aspartic acid residue or a glutamic acid residue.

More preferably, the position is selected from the group consisting of N4, L5, F8, L9, R11, S12, F15, Q16, Q18, K19, W22, Q23, G26, R27, Y30, M36, Q46, Q48, Q49, A68, R71, S75, T82, A89, N90, Y92, H93, N96, H97, K108, F111, L116, L120, K123, R124, G127, R128, L130, H131, K134, A135, H140, Y155, N158, R159, G162, Y163 and R165, such as from the group consisting of N4, L5, F8, S12, F15, Q16, K19, W22, Q23, R27, Y30, M36, Q46, Q48, Q49, R71, S75, T82, A89, Y92, H93, K108, F111, L116, K123, R124, G127, H131, K134, A135, Y155 and R165, still more preferably from the group consisting of N4, L5, F8, S12, F15, Q16, K19, W22, Q23, R27, Y30, Q46, Q48, Q49, S75, T82, A89, Y92, H93, K108, F111, L116, R124, G127, H131, K134, Y155 and R165, such as from the group consisting of L5, F8, S12, F15, Q16, K19, W22, Q23, Q48, Q49, Y92, H93, R124, G127, H131 and Y155, even more preferably from the group consisting of S12, Q16, K19, Q23, Q48, Q49, Y92, H93, R124, G127, H131 and Y155, such as from the group consisting of S12, Q16, K19, Q23, Q48, Y92, H93, R124, G127, H131 and Y155, in particular from the group consisting of S12, Q16, K19, Q23, Q48, H93 and H131, even more preferably from the group consisting of S12, Q16, K19, Q48, H93 and H131, and most preferably from the group consisting of Q16 and Q48.

Furthermore, in order to obtain a sufficient number of non-polypeptide moieties it is preferred that that least two aspartic acid residues or at least two glutamic acid residues be introduced, preferably in two positions selected from any of the above lists. Also, it is preferred that the conjugate according to this aspect comprises at least two first non-polypeptide moieties.

In case of removal of an amino acid residue, the amino acid sequence of the interferon β monomer differs from that of human wildtype interferon β in at least one removed aspartic acid or glutamic acid residue, such as 1-5 removed residues, in particular 1-4 or 1-3 removed aspartic acid or glutamic acid residues. The residue(s) to be removed, preferably by replacement, is selected from the group consisting of D34, D39, D54, D73, D110, E29, E42, E43, E53, E61, E81, E85, E103, E104, E107, E109, E137 and E149. The aspartic acid or glutamic acid residue(s) may be replaced with any other amino acid residue, but is preferably replaced by an arginine or a glutamine residue. While the first non-polypeptide moiety can be any non-polypeptide moiety with such property, it is presently preferred that the non-polypeptide moiety is a polymer molecule or an organic derivatizing agent having an acid group as an attachment group, in particular a polymer molecule such as PEG, and the conjugate is prepared, e.g., as described by Sakane and Pardridge, Pharmceutical Research, Vol. 14, No. 8, 1997, pp 1085-1091. Normally, for conjugation to an acid group the non-polypeptide moiety has a molecular weight of about 5 or 10 kDa.

Conjugate of the Invention Comprising a Second Non-polypeptide Moiety

In yet another aspect the invention relates to a conjugate exhibiting interferon β activity and comprising at least one polymer molecule and at least one sugar moiety covalently attached to a multimeric interferon β polypeptide. The multimeric polypeptide comprises at least one interferon β monomer, e.g., selected from wildtype human interferon β polypeptide or any variant thereof as disclosed herein.

For instance the interferon β monomer may comprise an amino acid sequence which differs from that of wild-type human interferon β in

a) at least one introduced and/or at least one removed amino acid residue comprising an attachment group for the polymer molecule, and/or

at least one introduced and/or at least one removed amino acid residue comprising an attachment group for the sugar moiety, i.e., an in vivo glycosylation site such as an N-glycosylation site.

In addition to a first non-polypeptide moiety (as described in the preceding sections), the conjugate of the invention may comprise a second non-polypeptide moiety of a different type as compared to the first non-polypeptide moiety. Preferably, in any of the above described conjugates wherein the first non-polypeptide moiety is, e.g., a polymer molecule such as PEG, a second non-polypeptide moiety is a sugar moiety, in particular an N-linked sugar moiety. While the second non-polypeptide moiety may be attached to a natural glycosylation site of the interferon β monomer, e.g., the N-linked glycosylation site defined by N80 in wildtype human interferon β, it is normally advantageous that at least one of the monomers comprises at least one additional glycosylation site. Such site is e.g., any of those described in the immediately preceding section entitled “Conjugate of the invention wherein the non-polypeptide moiety is a sugar moiety.” Furthermore, in case at least one additional glycosylation site is introduced this may be accompanied by removal of an existing glycosylation site as described below.

It will be understood that in order to obtain an optimal distribution of attached first and second non-polypeptide moieties, the interferon β monomer may be modified in the number and distribution of attachment groups for the first as well as the second non-polypeptide moiety so as to have e.g., at least one removed attachment group for the first non-polypeptide moiety and at least one introduced attachment group for the second non-polypeptide moiety or vice versa. Analogous considerations apply for the single chain multimeric interferon β polypeptide as such. For instance, the interferon β monomer or the multimeric interferon β polypeptide comprises at least two (e.g., 2-5) removed attachment groups for the first non-polypeptide moiety and at least one (e.g., 1-5) introduced attachment groups for the second non-polypeptide moiety or vice versa.

Of particular interest is a conjugate wherein the first non-polypeptide moiety is a polymer molecule such as PEG having lysine as an attachment group, and the second non-polypeptide moiety is an N-linked sugar moiety.

In a specific embodiment, the interferon monomer comprises one of the following sets of mutations:

K19R+K45R+Q49N+Q51T+F111N+R113T+K123R;

K19R+K45R+Q49N+Q51T+F111N+R113T;

K19R+K45R+Q49N+Q51T+K123R; or

C17S+K19R+K33R+K45R+Q49N+Q51T+D110F+F111N+R113T.

Conjugate of the Invention Wherein the Non-Polypeptide Moiety is a Sugar Moiety

In yet another aspect the invention relates to a conjugate exhibiting interferon β activity and comprising a multimeric interferon β polypeptide that comprises at least one interferon β monomer, the monomer comprises an amino acid sequence that differ from that of wild-type human interferon β in that a glycosylation site has been introduced or removed by way of introduction or removal of amino acid residue(s) constituting a part of a glycosylation site in a position that in wildtype human interferon β is occupied by a surface exposed amino acid residue.

When the conjugate of the invention comprises at least one sugar moiety attached to an in vivo glycosylation site, in particular an N-glycosylation site, this is either the natural N-glycosylation site of wild-type human interferon β at position N80, i.e., defined by amino acid residues N80, E81, T82 and I83, (in at least one of the monomers) or a new in vivo glycosylation site introduced into the multimeric interferon β polypeptide, i.e., in a monomer or peptide linker constituent thereof. The in vivo glycosylation site may be an O-glycosylation site, but is preferably an N-glycosylation site.

For instance, an in vivo glycosylation site is introduced into a position that in human wildtype interferon β is occupied by an amino acid residue exposed to the surface of the molecule, preferably with more than 25% of the side chain exposed to the solvent, in particular more than 50% exposed to the solvent (these positions are identified in the Methods section herein). The N-glycosylation site is introduced in such a way that the N-residue of said site is located in said position. Analogously, an O-glycosylation site is introduced so that the S or T residue making up such site is located in said position. Furthermore, in order to ensure efficient glycosylation it is preferred that the in vivo glycosylation site, in particular the N residue of the N-glycosylation site or the S or T residue of the O-glycosylation site, is located within the first 141 amino acid residues of the interferon β monomer, more preferably within the first 116 amino acid residues. Still more preferably, the in vivo glycosylation site is introduced into a position wherein only one mutation is required to create the site (i.e., where any other amino acid residues required for creating a functional glycosylation site is already present in the molecule).

Substitutions that lead to introduction of an additional N-glycosylation site at positions exposed at the surface of the interferon β monomer and occupied by amino acid residues having more than 25% of the side chain exposed to the surface include:

S2N+N4S/T, L6S/T, L5N+G7S/T, F8N+Q10S/T, L9N+R11S/T, R11N, R11N+S13T,

S12N+N14S/T, F15N+C17S/T, Q16N+Q18S/T, Q18N+L20S/T, K19N+L21S/T,

W22N+L24S/T, Q23N+H25S/T, G26N+L28S/T, R27N+E29S/T, L28S+Y30S/T,

Y30N+L32S/T, L32N+D34S/T, K33N+R35S/T, R35N+N37S/T, M36N+F38S/T, D39S/T,

D39N+P41S/T, E42N+I44S/T, Q43N+K45S/T, K45N+L47S/T, Q46N+Q48S/T,

L47N+Q49T/S, Q48N+F50S/T, Q49N+Q51S/T, Q51N+E53S/T, K52N+D54S/T,

L57N+I59S/T, Q64N+I66S/T, A68N+F70S/T, R71N+D73S/T, Q72N, Q72N+S74T, D73N,

D73N+S75T, S75N+T77S, S75N, S76N+G78S/T, E81N+I83S/T, T82N+V84S/T

E85N+L87S/T, L88S/T, A89N+V91S/T, Y92S/T, Y92N+Q94S/T, H93N+I95S/T, L98S/T,

H97N+K99S/T, K99N+V101S/T, T100N+L102S/T, E103N+K105S/T, E104N+L106S/T,

K105N+E107S/T, E107N+E109S/T, K108N+D110S/T, E109N+F111S/T, D110N+T112S,

D110N, F111N+R113S/T, R113N+K115S/T, G114N+L116S/T, K115N+M117S/T, L116N,

L116N+S118T, S119N+H212S/T, L120N+L122S/T, H121N+K123S/T, K123N+Y125S/T,

R124N+Y126S/T, G127N+I129S/T, R128N+L130S/T, L130N+Y132S/T, H131N+L133S/T,

K134N+K136S/T, A135N+E137S/T, K136N+Y138S/T, E137N, Y138N+H140S/T,

H140N+A142S/T, V148N+I150S/T, R152N+F154S/T, Y155N+I157S/T, L160S/T,

R159N+T161S, R159N, G162N+L164S/T, and Y163N+R165S/T (the numbering referring to that used in SEQ ID NO 2).

Substitutions that lead to introduction of an additional N-glycosylation site at positions exposed at the surface of the interferon β monomer having more than 50% of the side chain exposed to the surface include:

L6S/T, L5N+G7S/T, F8N+Q10S/T, L9N+R11S/T, S12N+N14S/T, F15N+C17S/T,

Q16N+Q18S/T, K19N+L21S/T, W22N+L24S/T, Q23N+H25S/T, G26N+L28S/T,

R27N+E29S/T, Y30N+L32S/T, K33N+R35S/T, R35N+N37S/T, M36N+F38S/T, D39S/T,

D39N+P41S/T, E42N+I44S/T, Q46N+Q48S/T, Q48N+F50S/T, Q49N+Q51S/T,

Q51N+E53S/T, K52N+D54S/T, L57N+I59S/T, R71N+D73S/T, D73N, D73N+S75T,

S75N+T77S, S75N, S76N+G78S/T, E81N+I83S/T, T82N+V84S/T, E85N+L87S/T,

A89N+V91S/T, Y92S/T, Y92N+Q94S/T, H93N+I95S/T, T100N+L102S/T, E103N+K105S/T,

E104N+L106S/T, E107N+E109S/T, K108N+D110S/T, D110N+T112S, D110N,

F111N+R113S/T, R113N+K115S/T, L116N, L116N+S118T, K123N+Y125S/T,

R124N+Y126S/T, G127N+I129S/T, H131N+L133S/T, K134N+K136S/T, A135N+E137S/T,

E137N, V148N+I150S/T, and Y155N+I157S/T (the numbering referring to that used in SEQ ID NO 2).

Among the substitutions mentioned in the above lists, those are preferred that have the N residue introduced among the 141 N-terminal amino acid residues, in particular among the 116 N-terminal amino acid residues.

Substitutions that lead to introduction of an N-glycosylation site by only one amino acid substitution include: L6S/T, R11N, D39S/T, Q72N, D73N, S75N, L88S/T, Y92S/T, L98S/T, D110N, L116N, E137N, R159N and L160S/T. Among these, a substitution is preferred that is selected from the group consisting of L6S/T, R11N, D39S/T, Q72N, D73N, S75N, L88S/T, Y92S/T, L98S/T, D110N and L116N, more preferably from the group consisting of L6S/T, D39S/T, D73N, S75N, L88S/T, D110N, L116N and E137N; and most preferably selected from the group consisting of L6S/T, D39S/T, D73N, S75N, L88S/T, D110N and L116N.

The presently most preferred interferon β monomer for use in this embodiment include at least one of the following substitutions:

S2N+N4T/S, L9N+R11T/S, R11N, S12N+N14T/S, F15N+C17S/T, Q16N+Q18T/S,

K19N+L21T/S, Q23N+H25T/S, G26N+L28T/S, R27N+E29T/S, L28N+Y30T/S, D39T/S,

K45N+L47T/S, Q46N+Q48T/S, Q48N+F50T/S, Q49N+Q51T/S, Q51N+E53T/S,

R71N+D73T/S, Q72N, D73N, S75N, S76N+G78T/S, L88T/S, Y92T/S, N93N+195T/S,

L98T/S, E103N+K105T/S, E104N+L106T/S, E107N+E109T/S, K108N+D110T/S, D110N,

F111N+R113T/S, or L116N, more preferably at least one of the following substitutions:

S2N+N4T, L9N+R11T, 49N+Q51T or F111N+R113T or R71N+D73T, in particular

49N+Q51T or F111N+R113T or R71N+D73T. For instance, the interferon β monomer comprises one of the following sets of substitutions:

Q49N+Q51T+F111N+R113T;

Q49N+Q51T+R71N+D73T+F111N+R113T;

C17S+Q49N+Q51T+D110F+F111N+R113T;

S2N+N4T+F111N+R113T;

S2N+N4T+Q49N+Q51T;

S2N+N4T+Q49N+Q51T+F111N+R113T;

S2N+N4T+L9N+R11T+Q49N+Q51T;

S2N+N4T+L9N+R11T+F111N+R113T;

S2N+N4T+L9N+R11T+Q49N+Q51T+F111N+R113T;

L9N+R11T+Q49N+Q51T;

L9N+R11T+Q49N+Q51T+F111N+R113T; or

L9N+R11T+F111N+R113T

It will be understood that in order to introduce a functional in vivo glycosylation site the amino acid residue in between the N-residue and the S/T residue is different from proline. Normally, the amino acid residue in between will be that occupying the relevant position in the amino acid sequence shown in SEQ ID NO 2. For instance, in a polypeptide comprising the substitutions Q49N+Q51S, position 50 is the position in between.

The multimeric interferon β polypeptide part of a conjugate of the invention may contain a single in vivo glycosylation site. However, in order to obtain efficient shielding of epitopes present on the surface of the polypeptide it is desirable that the polypeptide comprises more than one in vivo glycosylation site, in particular 2-7 in vivo glycosylation sites, such as 2, 3, 4, 5, 6 or 7 in vivo glycosylation sites. For instance, the interferon β monomer may comprise one additional glycosylation site, or may comprise two, three, four, five, six, seven or more introduced in vivo glycosylation sites, preferably introduced by one or more substitutions described in any of the above lists.

As indicated above, in addition to one or more introduced glycosylation sites, existing glycosylation sites may have been removed from the interferon β monomer. For instance, any of the above listed substitutions to introduce a glycosylation site may be combined with a substitution to remove the natural N-glycosylation site of human wild-type interferon β. For instance, the interferon β monomer may comprise a substitution of N80, e.g., one of the substitutions N80K/C/D/E, when a first non-polypeptide polypeptide is one having one of K, C, D, E as an attachment group. For instance, the interferon β monomer may comprise at least one of the following substitutions: S2N+N4T/S, L9N+R11T/S, R11N, S12N+N14T/S, F15N+C17S/T, Q16N+Q18T/S, K19N+L21T/S, Q23N+H25T/S, G26N+L28T/S, R27N+E29T/S, L28N+Y30T/S, D39T/S, K45N+L47T/S, Q46N+Q48T/S, Q48N+F50T/S, Q49N+Q51T/S, Q51N+E53T/S, R71N+D73T/S, Q72N, D73N, S75N, S76N+G78T/S, L88T/S, Y92T/S, N93N+195T/S, L98T/S, E103N+K105T/S, E104N+L106T/S, E107N+E109T/S, K108N+D110T/S, D110N, F111N+R113T/S, or L116N in combination with N80K/C/D/E. More specifically, the interferon β monomer may comprise the substitution: Q49N+Q51T or F111N+R113T or R71N+D73T, in particular Q49N+Q51T+F111N+R113T or Q49N+Q51T+R71N+D73T+F111N+R111T, in combination with N80K/C/D/E.

A single chain multimeric polypeptide comprising any of the interferon β monomers disclosed in the present section having introduced and/or removed at least one glycosylation site, such as the monomer comprising the substitutions Q48N+F50T/S, Q48N+F50T/S+F111N+R113T/S, Q49N+Q51T/S, F111N+R113T/S, or Q49N+Q51 T/S+F111N+R113T/S, may further be conjugated to a polymer molecule, such as PEG, or any other non-polypeptide moiety. For this purpose the conjugation may be achieved by use of attachment groups already present in either of the monomers or attachment groups may have been introduced and/or removed, in particular such that a total of 1-6, in particular 3-4 or 1, 2, 3, 4, 5, or 6 attachment groups are available for conjugation.

In particular, a glycosylated single chain multimeric interferon β polypeptide may be conjugated to a non-polypeptide moiety via a lysine attachment group, and one or more lysine residues of at least one monomer may have been removed, e.g., when the monomer is an interferon β monomer, by any of the substitutions mentioned in the section entitled “Conjugate of the invention, wherein the non-polypeptide moiety is a molecule which has lysine as an attachment group,” in particular the substitutions K19R+K45R+K123R. Alternatively or additionally, a lysine residue may have been introduced, e.g., by any of the substitutions mentioned in said section, in particular the substitution R71K. Accordingly, one specific conjugate of the invention is one, which comprises a glycosylated multimeric interferon β polypeptide, comprising at least one of the interferon β monomer that comprises the mutations Q49N+Q51T+F111N+R113T+K19R+K45R+K123R or Q49N+Q51T+F111N+R113T+K19R +K45R+K123R+R71K further conjugated to PEG. The glycosylated polypeptide part of said conjugate is favourably produced in CHO cells and PEGylated subsequent to purification using e.g., SS-PEG, NPC-PEG, aldehyd-PEG, mPEG-SPA, mPEG-SCM, mPEG-BTC from Shearwater Polymers, Inc, SC-PEG from Enzon, Inc., tresylated mPEG as described in U.S. Pat. No. 5,880,255, or oxycarbonyl-oxy-N-dicarboxyimide-PEG (U.S. Pat. No. 5,122,614).

Alternatively to PEGylation via a lysine group, the glycosylated conjugate according to this embodiment may be PEGylated via a cysteine group as described in the section entitled “Conjugate of the invention, wherein the non-polypeptide moiety is a molecule that has cysteine as an attachment group” (for this purpose the interferon β monomer may comprise, e.g., at least one of the mutations N80C, R71C and C17S), via an acid group as described in the section entitled “Conjugation of the invention wherein the non-polypeptide moiety binds to an acid group,” or via any other suitable group.

Other Conjugates of the Invention

In addition to the introduction and/or removal of amino acid residues comprising an attachment group for the non-polypeptide moiety of choice (as described in any of the sections above entitled “Conjugate of the invention . . . ”) the interferon β monomer may contain further substitutions. A preferred example is a substitution of any of the residues, M1, C17, N80 or V101, e.g., one or more of the following substitutions: C17S; N80K/C/D/E; V101Y/W/F/, H; a deletion of M1; or M1K.

Non-polypeptide Moiety of the Conjugate of the Invention

As indicated further above the non-polypeptide moiety of the conjugate of the invention is preferably selected from the group consisting of a polymer molecule, a lipophilic compound, a sugar moiety (by way of in vivo glycosylation) and an organic derivatizing agent. All of these agents may confer desirable properties to the multimeric polypeptide part of the conjugate, in particular reduced immunogenicity and/or increased functional in vivo half-life and/or increased serum half-life. The multimeric polypeptide part of the conjugate may be conjugated to only one type of non-polypeptide moiety, but may also be conjugated to two or more different types of non-polypeptide moieties, e.g., to a polymer molecule and a sugar moiety, to a lipophilic group and a sugar moiety, to an organic derivating agent and a sugar moiety, to a lipophilic group and a polymer molecule, etc. The conjugation to two or more different non-polypeptide moieties may be done simultaneous or sequentially. The choice of non-polypeptide moiety/ies, e.g., depends on the effect desired to be achieved by the conjugation. For instance, sugar moieties have been found particularly useful for reducing immunogenicity, whereas polymer molecules such as PEG are of particular use for increasing functional in vivo half-life and/or serum half-life. Using a polymer molecule as a first non-polypeptide moiety and a sugar moiety as a second non-polypeptide moiety may result in reduced immunogenicity and increased functional in vivo or serum half-life.

Methods of Preparing a Conjugate of the Invention

In the following sections “Conjugation to a lipophilic compound,” “Conjugation to a polymer molecule,” “Conjugation to a sugar moiety” and “Conjugation to an organic derivatizing agent” conjugation to specific types of non-polypeptide moieties is described.

Conjugation to a Lipophilic Compound

For conjugation to a lipophilic compound the following polypeptide groups may function as attachment groups: the N-terminal or C-terminal of the polypeptide, the hydroxy groups of the amino acid residues Ser, Thr or Tyr, the E-amino group of Lys, the SH group of Cys or the carboxyl group of Asp and Glu. The polypeptide and the lipophilic compound may be conjugated to each other, either directly or by use of a linker. The lipophilic compound may be a natural compound such as a saturated or unsaturated fatty acid, a fatty acid diketone, a terpene, a prostaglandin, a vitamine, a carotenoide or steroide, or a synthetic compound such as a carbon acid, an alcohol, an amine and sulphonic acid with one or more alkyl-, aryl-, alkkenyl- or other multiple unsaturated compounds. The conjugation between the polypeptide and the lipophilic compound, optionally through a linker may be done according to methods known in the art, e.g., as described by Bodanszky in Peptide Synthesis, John Wiley, New York, 1976 and in WO 96/12505.

Conjugation to a Polymer Molecule

The polymer molecule to be coupled to the polypeptide may be any suitable polymer molecule, such as a natural or synthetic homo-polymer or heteropolymer, typically with a molecular weight in the range of 300-100,000 Da, such as 300-20,000 Da, more preferably in the range of 500-10,000 Da, even more preferably in the range of 500-5000 Da.

Examples of homo-polymers include a polyol (i.e., poly-OH), a polyamine (i.e., poly-NH₂) and a polycarboxylic acid (i.e., poly-COOH). A hetero-polymer is a polymer, which comprises one or more different coupling groups, such as, e.g., a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-malic acid anhydride, dextran including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is the preferred polymer molecule to be used, since it has only few reactive groups capable of cross-linking compared, e.g., to polysaccharides such as dextran, and the like. In particular, monofunctional PEG, e.g monomethoxypolyethylene glycol (mPEG), is of interest since its coupling chemistry is relatively simple (only one reactive group is available for conjugating with attachment groups on the polypeptide ). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e., with reactive functional groups (examples of which include primary amino groups, hydrazide (HZ), thiol, succinate (SUC), succinimidyl succinate (SS), succinimidyl succinamide (SSA), succinimidyl proprionate (SPA), succinimidy carboxymethylate (SCM), benzotriazole carbonate (BTC), N-hydroxysuccinimide (NHS), aldehyde, nitrophenylcarbonate (NPC), and tresylate (TRES)). Suitably activated polymer molecules are commercially available, e.g., from Shearwater Polymers, Inc., Huntsville, Ala., USA. Alternatively, the polymer molecules can be activated by conventional methods known in the art, e.g., as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which references are incorporated herein by reference. Furthermore, the following publications, incorporated herein by reference, disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94118247, WO 94/28024, WO 95/00162, WO 95/11924, W095/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g., as described in the following references (which also describe suitable methods for activation of polymer molecules): Harris and Zalipsky, eds., Poly(ethylene glycol) Chemistry and Biological Applications, AZC, Washington; R. F. Taylor, (1991), “Protein immobilisation. Fundamental and applications,” Marcel Dekker, N.Y.; S. S. Wong, (1992), “Chemistry of Protein Conjugation and Crosslinking,” CRC Press, Boca Raton; G. T. Hermanson et al., (1993), “Immobilized Affinity Ligand Techniques,” Academic Press, N.Y.). The skilled person will be aware that the activation method and/or conjugation chemistry to be used depends on the attachment group(s) of the interferon β polypeptide as well as the functional groups of the polymer (e.g., being amino, hydroxyl, carboxyl, aldehyde or sulfydryl). The PEGylation may be directed towards conjugation to all available attachment groups on the polypeptide (i.e., such attachment groups that are exposed at the surface of the polypeptide) or may be directed towards specific attachment groups, e.g., the N-terminal amino group (U.S. Pat. No. 5,985,265). Furthermore, the conjugation may be achieved in one step or in a stepwise manner (e.g., as described in WO 99/55377).

It will be understood that the PEGylation is designed so as to produce the optimal molecule with respect to the number of PEG molecules attached, the size and form (e.g., whether they are linear or branched) of such molecules, and where in the polypeptide such molecules are attached. For instance, the molecular weight of the polymer to be used may be chosen on the basis of the desired effect to be achieved. For instance, if the primary purpose of the conjugation is to achieve a conjugate having a high molecular weight (e.g., to reduce renal clearance) it is usually desirable to conjugate as few high Mw polymer molecules as possible to obtain the desired molecular weight. When a high degree of epitope shielding is desirable this may be obtained by use of a sufficiently high number of low molecular weight polymer (e.g., with a molecular weight of about 5,000 Da) to effectively shield all or most epitopes of the polypeptide. For instance, 2-8, such as 3-6 such polymers may be used.

In connection with conjugation to only a single attachment group on the protein (as described in U.S. Pat. No. 5,985,265), it may be advantageous that the polymer molecule, which may be linear or branched, has a high molecular weight, e.g., about 20 kDa.

Normally, the polymer conjugation is performed under conditions aiming at reacting all available polymer attachment groups with polymer molecules. Typically, the molar ratio of activated polymer molecules to polypeptide is 1000-1, in particular 200-1, preferably 100-1, such as 10-1 or 5-1 in order to obtain optimal reaction. However, also equimolar ratios may be used.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378.

Subsequent to the conjugation residual activated polymer molecules are blocked according to methods known in the art, e.g., by addition of primary amine to the reaction mixture, and the resulting inactivated polymer molecules removed by a suitable method.

Covalent in vitro coupling of a carbohydrate moiety to amino acid residues of interferon β may be used to modify or increase the number or profile of carbohydrate substituents. Depending on the coupling mode used, the carbohydrate(s) may be attached to a) arginine and histidine (Lundblad and Noyes, Chemical Reagents for Protein Modification, CRC Press Inc. Boca Raton, Fla.), b) free carboxyl groups (e.g., of the C-terminal amino acid residue, asparagine or glutamine), c) free sulfhydryl groups such as that of cysteine, d) free hydroxyl groups such as those of serine, threonine, tyrosine or hydroxyproline, e) aromatic residues such as those of phenylalanine or tryptophan or f) the amide group of glutamine. These amino acid residues constitute examples of attachment groups for a carbohydrate moiety, which may be introduced and/or removed in the interferon β polypeptide. Suitable methods of in vitro coupling are described in WO 87/05330 and in Aplin et al., CRC Crit Rev. Biochem., pp. 259-306, 1981. The in vitro coupling of carbohydrate moieties or PEG to protein- and peptide-bound Gln-residues can also be carried out by transglutaminases (TGases), e.g., as described by Sato et al., 1996 Biochemistry 35, 13072-13080 or in EP 725145

Coupling to a Sugar Moiety

In order to achieve in vivo glycosylation of an interferon β polypeptide that has been modified by introduction of one or more glycosylation sites (see the section “Conjugates of the invention wherein the non-polypeptide moiety is a sugar moiety”), the nucleotide sequence encoding the polypeptide part of the conjugate must be inserted in a glycosylating, eucaryotic expression host. The expression host cell may be selected from fungal (filamentous fungal or yeast), insect, mammalian animal cells, from transgenic plant cells or from transgenic animals. Furthermore, the glycosylation may be achieved in the human body when using a nucleotide sequence encoding the polypeptide part of a conjugate of the invention or a polypeptide of the invention in gene therapy. In one embodiment the host cell is a mammalian cell, such as an CHO cell, BHK or HEK cell, e.g., HEK293, or an insect cell, such as an SF9 cell, or a yeast cell, e.g., Saccharomyces cerevisiae, Pichia pastoris or any other suitable glycosylating host, e.g., as described further below. In a particular embodiment the host cell is a CHO-K1 cell. Optionally, sugar moieties attached to the interferon β polypeptide by in vivo glycosylation are further modified by use of glycosyltransferases, e.g., using the glycoAdvance™ technology marketed by Neose, Horsham, Pa., USA. Thereby, it is possible to, e.g., increase the sialyation of the glycosylated interferon β polypeptide following expression and in vivo glycosylation by CHO cells.

Coupling to an Organic Derivatizing Agent

Covalent modification of the single chain multimeric interferon β polypeptide may be performed by reacting (an) attachment group(s) of the polypeptide with an organic derivatizing agent. Suitable derivatizing agents and methods are well known in the art. For example, cysteinyl residues most commonly are reacted with (x-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonateat pH 5.5-7.0 because this agent is relatively specific for the histidyl side chain. Para-bromophenacyl bromide also is useful; the reaction is preferably performed in 0.1M sodium cacodylate at pH 6.0.Lysinyl and amino terminal residues are reacted with succinic or other carboxylic acid anhydrides. Derivatization with these agents has the effect of reversing the charge of the lysinyl residues. Other suitable reagents for derivatizing cc-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine guanidino group. Carboxyl side groups (aspartyl or glutamyl or C-terminal amino acid residue) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Blocking of Functional Site

It has been reported that excessive polymer conjugation can lead to a loss of activity of the interferon β polypeptide to which the polymer is conjugated. This problem can be eliminated, e.g., by removal of attachment groups located at the functional site or by blocking the functional site prior to conjugation. These latter strategies constitute further embodiments of the invention (the first strategy being exemplified further above, e.g., by removal of lysine residues which may be located close to a functional site). More specifically, according to the second strategy the conjugation between the single chain multimeric interferon β polypeptide and the non-polypeptide moiety is conducted under conditions where the functional site of the polypeptide is blocked by a helper molecule capable of binding to the functional site of the polypeptide. Preferably, the helper molecule is one, which specifically recognizes a functional site of the polypeptide, such as a receptor, in particular the type I interferon receptor. Alternatively, the helper molecule may be an antibody, in particular a monoclonal antibody recognizing the interferon β polypeptide. In particular, the helper molecule may be a neutralizing monoclonal antibody.

The polypeptide is allowed to interact with the helper molecule before effecting conjugation. This ensures that the functional site of the polypeptide is shielded or protected and consequently unavailable for derivatization by the non-polypeptide moiety such, as a polymer. Following its elution from the helper molecule, the conjugate between the non-polypeptide moiety and the polypeptide can be recovered with at least a partially preserved functional site.

The subsequent conjugation of the polypeptide having a blocked functional site to a polymer, a lipophilic compound, an organic derivatizing agent or any other compound is conducted in the normal way, e.g., as described in the sections above entitled “Conjugation to . . . ”

Irrespectively of the nature of the helper molecule to be used to shield the functional site of the polypeptide from conjugation, it is desirable that the helper molecule is free from or comprises only a few attachment groups for the non-polypeptide moiety of choice in part(s) of the molecule, where the conjugation to such groups will hamper the desorption of the conjugated polypeptide from the helper molecule. Hereby, selective conjugation to attachment groups present in non-shielded parts of the polypeptide can be obtained and it is possible to reuse the helper molecule for repeated cycles of conjugation. For instance, if the non-polypeptide moiety is a polymer molecule such as PEG, which has the epsilon amino group of a lysine or N-terminal amino acid residue as an attachment group, it is desirable that the helper molecule is substantially free from conjugatable epsilon amino groups, preferably free from any epsilon amino groups. Accordingly, in a preferred embodiment the helper molecule is a protein or peptide capable of binding to the functional site of the polypeptide, which protein or peptide is free from any conjugatable attachment groups for the non-polypeptide moiety of choice.

In a further embodiment the helper molecule is first covalently linked to a solid phase such as column packing materials, for instance Sephadex or agarose beads, or a surface, e.g., reaction vessel. Subsequently, the polypeptide is loaded onto the column material carrying the helper molecule and conjugation carried out according to methods known in the art, e.g., as described in the sections above entitled “Conjugation to . . . ” This procedure allows the polypeptide conjugate to be separated from the helper molecule by elution. The polypeptide conjugate is eluated by conventional techniques under physico-chemical conditions that do not lead to a substantive degradation of the polypeptide conjugate. The fluid phase containing the polypeptide conjugate is separated from the solid phase to which the helper molecule remains covalently linked. The separation can be achieved in other ways: For instance, the helper molecule may be derivatised with a second molecule (e.g., biotin) that can be recognized by a specific binder (e.g., streptavidin). The specific binder may be linked to a solid phase thereby allowing the separation of the polypeptide conjugate from the helper molecule-second molecule complex through passage over a second helper-solid phase column which will retain, upon subsequent elution, the helper molecule-second molecule complex, but not the polypeptide conjugate. The polypeptide conjugate may be released from the helper molecule in any appropriate fashion. De-protection may be achieved by providing conditions in which the helper molecule dissociates from the functional site of the interferon β to which it is bound. For instance, a complex between an antibody to which a polymer is conjugated and an anti-idiotypic antibody can be dissociated by adjusting the pH to an acid or alkaline pH.

Methods of Preparing a Single Chain Multimeric Interferon β polypeptide for use in the Invention

The multimeric polypeptide described herein optionally in glycosylated form, may be produced by any suitable method known in the art. Such methods include constructing a nucleotide sequence encoding the polypeptide and expressing the sequence in a suitable transformed or transfected host. However, polypeptides of the invention may be produced, albeit less efficiently, by chemical synthesis or a combination of chemical synthesis or a combination of chemical synthesis and recombinant DNA technology. The invention thus encompasses a method for preparing a single-chain multimeric polypeptide or polypeptide conjugate as disclosed herein, comprising culturing a recombinant host cell comprising a single nucleotide sequence encoding said polypeptide in a suitable culture medium under conditions permitting expression of the nucleotide sequence, and recovering the resulting polypeptide from the cell culture, followed, where appropriate, by reacting the polypeptide with a polymer molecule under conditions permitting conjugation to take place so as to result in a polymer-polypeptide conjugate, and recovering the conjugate.

In a further aspect the invention relates to a nucleotide sequence encoding a single chain multimeric interferon beta polypeptide of the invention.

The nucleotide sequence of the invention encoding a single chain multimeric interferon β polypeptide may be constructed by isolating or synthesizing a nucleotide sequence encoding the single-chain nucleotide sequence (or a parent thereof and then changing the nucleotide sequence so as to effect introduction (i.e., insertion or substitution) or deletion (i.e., removal or substitution) of the relevant amino acid residue(s)).

The nucleotide sequence is conveniently modified by site-directed mutagenesis in accordance with well-known methods, see, e.g., Mark et al., “Site-specific Mutagenesis of the Human Fibroblast Interferon Gene,” Proc. Natl. Acad. Sci. USA, 81, pp. 5662-66 (1984); and U.S. Pat. No. 4,588,585.

Alternatively, the nucleotide sequence is prepared by chemical synthesis, e.g., by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide may be synthesized and assembled by PCR, ligation or ligation chain reaction (LCR). The individual oligonucleotides typically contain 5′ or 3′ overhangs for complementary assembly.

Once assembled (by synthesis, site-directed mutagenesis or another method), the nucleotide sequence encoding the multimeric polypeptide is inserted into a recombinant vector and operably linked to control sequences necessary for expression of the polypeptide in the desired transformed host cell.

In a further aspect the invention relates to an expression vector harbouring a nucleotide sequence encoding a single chain multimeric interferon beta polypeptide of the invention.

It should of course be understood that not all vectors and expression control sequences function equally well to express the nucleotide sequence encoding a multimeric polypeptide described herein. Neither will all hosts function equally well with the same expression system. However, one of skill in the art may make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and the ease of purification of the products coded for by the nucleotide sequence.

The recombinant vector may be an autonomously replicating vector, i.e., a vector which exists as an extrachromosomal entity, the replication of which is independent of chromosomal replication, e.g., a plasmid. Alternatively, the vector is one which, when introduced into a host cell, is integrated into the host cell genome and replicated together with the chromosome(s) into which it has been integrated.

The vector is preferably an expression vector, in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts, include, for example, vectors comprising expression control sequences from SV40, bovine papilloma virus, adenovirus and cytomegalovirus. Specific vectors are, e.g., pCDNA3.1(+)Hyg (Invitrogen, Carlsbad, Calif., USA) and pCI-neo (Stratagene, La Jola, Calif., USA). Useful expression vectors for bacterial hosts include known bacterial plasmids, such as plasmids from E. coli, including pBR322, pET3a and pET12a (both from Novagen Inc., Wis., USA), wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2μ plasmid and derivatives thereof, the POT1 vector (U.S. Pat. No. 4,931,373), the pJSO37 vector described in (Okkels, Ann. New York Acad. Sci. 782, 202-207, 1996) and pPICZ A, B or C (Invitrogen). Useful vectors for insect cells include pVL941, pBG311 (Cate et al., “Isolation of the Bovine and Human Genes for Mullerian Inhibiting Substance And Expression of the Human Gene In Animal Cells,” Cell, 45, pp. 685-98 (1986), pBluebac 4.5 and pMelbac (both available from Invitrogen).

Other vectors for use in this invention include those that allow the nucleotide sequence encoding the polypeptide variant to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrofolate Reductase cDNA Gene: Analysis Of Signals Utilized For Efficient Expression,” Mol. Cell. Biol., 2, pp. 1304-19 (1982)) and glutamine synthetase (“GS”) amplification (see, e.g., U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g., a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g., ampicillin, kanamycin, tetracyclin, chloramphenicol, neomycin, hygromycin or methotrexate. For filamentous fungi, selectable markers include amdS, pyrG, arcB, niaD, sC.

The term “control sequences” is defined herein to include all components, which are necessary or advantageous for the expression of the polypeptide of the invention. Each control sequence may be native or foreign to the nucleic acid sequence encoding the polypeptide. Such control sequences include, but are not limited to, a leader, polyadenylation sequence, propeptide sequence, promoter, enhancer or upstream activating sequence, signal peptide sequence, and transcription terminator. At a minimum, the control sequences include a promoter.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g., the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus Elb region polyadenylation signals and the Kozak consensus sequence (Kozak, M. J Mol Biol Aug. 20, 1987; 196(4):947-50).

In order to improve expression in mammalian cells a synthetic intron may be inserted in the 5′ untranslated region of the nucleotide sequence encoding the polypeptide of interest. An example of a synthetic intron is the synthetic intron from the plasmid pCI-Neo (available from Promega Corporation, Wis., USA).

Examples of suitable control sequences for directing transcription in insect cells include the polyhedrin promoter, the P10 promoter, the Autographa californica polyhedrosis virus basic protein promoter, the baculovirus immediate early gene 1 promoter and the baculovirus 39K delayed-early gene promoter, and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydogenase genes, the ADH2-4c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator.

Examples of suitable control sequences for use in bacterial host cells include promoters of the lac system, the trp system, the TAC or TRC system and the major promoter regions of phage lambda.

The nucleotide sequence of the invention encoding a multimeric polypeptide, whether prepared by site-directed mutagenesis, synthesis or other methods, may or may not also include a nucleotide sequence that encode a signal peptide. The signal peptide is present when the polypeptide is to be secreted from the cells in which it is expressed. Such signal peptide, if present, should be one recognized by the cell chosen for expression of the polypeptide. The signal peptide may be homologous (e.g., be that normally associated with human interferon β or heterologous (i.e., originating from another source than human interferon β to the polypeptide or may be homologous or heterologous to the host cell, i.e., be a signal peptide normally expressed from the host cell or one which is not normally expressed from the host cell. Accordingly, the signal peptide may be prokaryotic, e.g., derived from a bacterium such as E. coli, or eukaryotic, e.g., derived from a mammalian, or insect or yeast cell.

The presence or absence of a signal peptide will, e.g., depend on the expression host cell used for the production of the polypeptide, the protein to be expressed (whether it is an intracellular or extracellular protein) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. WO 90/05783), such as the lepidopteran Manduca sexta adipokinetic hormone precursor, (cf U.S. Pat. No. 5,023,328), the honeybee melittin (Invitrogen), ecdysteroid UDPglucosyltransferase (egt) (Murphy et al., Protein Expression and Purification 4, 349-357 (1993) or human pancreatic lipase (hpl) (Methods in Enzymology 284, pp. 262-272, 1997).

A preferred signal peptide for use in mammalian cells is that of human interferon β apparent from the examples hereinafter or the murine Ig kappa light chain signal peptide (Coloma, M (1992) J. Imm. Methods 152:89-104). For use in yeast cells suitable signal peptides have been found to be the α-factor signal peptide from S. cereviciae. (cf. U.S. Pat. No. 4,870,008), the signal peptide of mouse salivary amylase (cf. O. Hagenbuchle et al.,Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), and the yeast aspartic protease 3 (YAP3) signal peptide (cf. M. Egel-Mitani et al., Yeast 6, 1990, pp. 127-137).

Any suitable host may be used to produce the interferon β polypeptide, including bacteria, fungi (including yeasts), plant, insect, mammal, or other appropriate animal cells or cell lines, as well as transgenic animals or plants. Examples of bacterial host cells include grampositive bacteria such as strains of Bacillus, e.g., B. brevis or B. subtilis, Pseudomonas or Streptomyces, or gramnegative bacteria, such as strains of E. coli. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

In a further aspect the invention relates to a host cell comprising a nucleotide sequence encoding a single chain multimeric interferon beta polypeptide of the invention.

In a further aspect the invention relates to a host cell comprising an expression vector harbouring a nucleotide sequence encoding a single chain multimeric interferon beta polypeptide of the invention.

Examples of suitable filamentous fungal host cells include strains of Aspergillus, e.g., A. oryzae, A. niger, or A. nidulans, Fusarium or Trichoderma. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Examples of suitable yeast host cells include strains of Saccharomyces, e.g., S. cerevisiae, Schizosaccharomyces, Klyveromyces, Pichia, such as P. pastoris or P. methanolica, Hansenula, such as H. Polymorpha or Yarrowia. Methods for transforming yeast cells with heterologous DNA and producing heterologous polypeptides therefrom are disclosed by Clontech Laboratories, Inc, Palo Alto, Calif., USA (in the product protocol for the Yeastmaker™ Yeast Tranformation System Kit), and by Reeves et al., FEMS Microbiology Letters 99 (1992) 193-198, Manivasakam and Schiestl, Nucleic Acids Research, 1993, Vol. 21, No. 18, pp. 4414-4415 and Ganeva et al., FEMS Microbiology Letters 121 (1994) 159-164.

Examples of suitable insect host cells include a Lepidoptora cell line, such as Spodoptera frugiperda (Sf9 or Sf21) or Trichoplusioa ni cells (High Five) (U.S. Pat. No. 5,077,214). Transformation of insect cells and production of heterologous polypeptides therein may be performed as described by Invitrogen.

Examples of suitable mammalian host cells include Chinese hamster ovary (CHO) cell lines, (e.g., CHO-K1; ATCC CCL-61), Green Monkey cell lines (COS) (e.g., COS 1 (ATCC CRL-1650), COS 7 (ATCC CRL-1651)); mouse cells (e.g., NS/O), Baby Hamster Kidney (BHK) cell lines (e.g., ATCC CRL-1632 or ATCC CCL-10), and human cells (e.g., HEK 293 (ATCC CRL-1573)), as well as plant cells in tissue culture. Additional suitable cell lines are known in the art and available from public depositories such as the American Type Culture Collection, Rockville, Md. Also, the mammalian cell, such as a CHO cell, may be modified to express sialyltransferase, e.g., 1,6-sialyltransferase, e.g., as described in U.S. Pat. No. 5,047,335, in order to provide improved glycosylation of the multimeric polypeptide.

All of the above mentioned host cells are each independently considered an embodiment of the host cell of the invention.

In a particular embodiment the host cell is selected from a CHO, BHK, HEK293 cell or an SF9 cell. In one embodiment the host cell is a CHO-K1 cell.

Methods for introducing exogeneous DNA into mammalian host cells include calcium phosphate-mediated transfection, electroporation, DEAE-dextran mediated transfection, liposome-mediated transfection, viral vectors and the transfection methods described by Life Technologies Ltd, Paisley, UK using Lipofectamin 2000 and Roche Diagnostics Corporation, Indianapolis, USA using FuGENE 6. These methods are well known in the art and e.g., described by Ausbel et al. (eds.), 1996, Current Protocols in Molecular Biology, John Wiley & Sons, New York, USA. The cultivation of mammalian cells are conducted according to established methods, e.g., as disclosed in (Animal Cell Biotechnology, Methods and Protocols, Edited by Nigel Jenkins, 1999, Human Press Inc, Totowa, N.J., USA and Harrison M A and Rae I F, General Techniques of Cell Culture, Cambridge University Press 1997).

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The resulting polypeptide may be recovered by methods known in the art. For example, the polypeptide may be recovered from the nutrient medium by conventional procedures including, but not limited to, centrifugation, filtration, extraction, spray drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989). Specific methods for purifying polypeptides exhibiting interferon β activity are disclosed in U.S. Pat. Nos. 4,289,689, 4,359,389, 4,172,071, 4,551,271, 5,244,655, 4,485,017, 4,257,938 and 4,541,952. A specific purification method is based on immunoaffinity purification (see, e.g., Okamura et al., “Human Fibroblastoid Interferon: Immunosorbent Column Chromatography And N-Terminal Amino Acid Sequence,” Biochem., 19, pp. 3831-35 (1980)). Furthermore, purification may be based on the use of IFNAR 1 and/or IFNAR 2, in particular IFNAR 2.

The biological activity of the interferon β polypeptides can be assayed by any suitable method known in the art. Such assays include antibody neutralization of antiviral activity, induction of protein kinase, oligoadenylate 2,5-A synthetase or phosphodiesterase activities, as described in EP 41313 B1. Such assays also include immunomodulatory assays (see, e.g., U.S. Pat. No. 4,753,795), growth inhibition assays, and measurement of binding to cells that express interferon receptors. Specific assays for determining the biological activity of polypeptides or conjugates of the invention are disclosed in the Materials and Methods section hereinafter.

Other Methods of the Invention

In a still further aspect the invention relates to a method of increasing functional in vivo half-life and/or serum half-life of an interferon β polypeptide, which method comprises expressing the interferon β polypeptide in single chain multimeric form (as described herein) in a suitable host cell, and if relevant, conjugating the interferon β polypeptide to the non-polypeptide moiety.

In a still further aspect the invention relates to a method for preparing a conjugate of the invention, wherein the multimeric interferon β polypeptide is reacted with the non-polypeptide moiety to which it is to be conjugated under conditions conducive for the conjugation to take place, and the conjugate is recovered.

Pharmaceutical Compositition and Uses of a Conjugate of the Invention

The multimeric interferon β polypeptide or the conjugate of the invention is administered at a dose approximately paralleling that employed in therapy with human interferon β such as Avonex, Rebif and Betaseron, or a higher dosis. The exact dose to be administered depends on the circumstances. Normally, the dose should be capable of preventing or lessening the severity or spread of the condition or indication being treated. It will be apparent to those of skill in the art that an effective amount of a polypeptide, conjugate or composition of the invention depends, inter alia, upon the disease, the dose, the administration schedule, whether the polypeptide or conjugate or composition is administered alone or in conjunction with other therapeutic agents, the serum half-life of the compositions, and the general health of the patient.

The polypeptide or conjugate of the invention can be used “as is” and/or in a salt form thereof. Suitable salts include, but are not limited to, salts with alkali metals or alkaline earth metals, such as sodium, potassium, lithium, calcium and magnesium, as well as, e.g., zinc salts. These salts or complexes may by present as a crystalline and/or amorphous structure.

The polypeptide or conjugate of the invention is preferably administered in a composition including a pharmaceutically acceptable carrier or excipient. “Pharmaceutically acceptable” means a carrier or excipient that does not cause any untoward effects in patients to whom it is administered. Such pharmaceutically acceptable carriers and excipients are well known in the art.

The polypeptide or conjugate of the invention can be formulated into pharmaceutical compositions by well-known methods. Suitable formulations are described in U.S. Pat. No. 5,183,746, Remington's Pharmaceutical Sciences by E. W. Martin, 18^{th} edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]).

In a further aspect the invention relates to a pharmaceutical composition comprising a single chain multimeric interferon beta polypeptide of the invention and a pharmaceutically acceptable diluent, carrier or adjuvant.

In a further aspect the invention relates to a pharmaceutical composition comprising a conjugate of a single chain multimeric interferon beta polypeptide of the invention and a pharmaceutically acceptable diluent, carrier or adjuvant.

The pharmaceutical composition of the polypeptide or conjugate of the invention may be formulated in a variety of forms, including liquid, gel, lyophilized, pulmonary dispersion, or any other suitable form, e.g., as a compressed solid. The preferred form will depend upon the particular indication being treated and will be apparent to one of skill in the art.

The pharmaceutical composition containing the polypeptide or conjugate of the invention may be administered orally, intravenously, intracerebrally, intramuscularly, intraperitoneally, intradermally, subcutaneously, intranasally, intrapulmonary, by inhalation, or in any other acceptable manner, e.g., using PowderJect or ProLease technology. The preferred mode of administration will depend upon the particular indication being treated and will be apparent to one of skill in the art.

Parenterals

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives are added to retard microbial growth, and are typically added in amounts of about 0.2%-1% (w/v). Suitable preservatives for use with the present invention include phenol, benzyl alcohol, meta-cresol, methyl paraben, propyl paraben, octadecyldimethylbenzyl ammonium chloride, benzalkonium halides (e.g., benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, omithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also know n as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g., starch), chelating agents (e.g., EDTA), antioxidants (e.g., ascorbic acid, methionine, vitamin E) and cosolvents. The active ingredient may also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

Parenteral formulations to be used for in vivo administration must be sterile. This is readily accomplished, for example, by filtration through sterile filtration membranes.

Sustained Release Preparations

Suitable examples of sustained-release preparations include semi-permeable matrices of solid hydrophobic polymers containing the polypeptide or conjugate, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the ProLease® technology or Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Pulmonary Delivery

Conjugate formulations suitable for use with a nebulizer, either jet or ultrasonic, will typically comprise the conjugate dissolved in water at a concentration of, e.g., about 0.01 to 25 mg of conjugate per mL of solution, preferably about 0.1 to 10 mg/mL. The formulation may also include a buffer and a simple sugar (e.g., for protein stabilization and regulation of osmotic pressure), and/or human serum albumin ranging in concentration from 0.1 to 10 mg/ml. Examples of buffers that may be used are sodium acetate, citrate and glycine. Preferably, the buffer will have a composition and molarity suitable to adjust the solution to a pH in the range of 3 to 9. Generally, buffer molarities of from 1 mM to 50 mM are suitable for this purpose. Examples of sugars which can be utilized are lactose, maltose, mannitol, sorbitol, trehalose, and xylose, usually in amounts ranging from 1% to 10% by weight of the formulation.

The nebulizer formulation may also contain a surfactant to reduce or prevent surface induced aggregation of the protein caused by atomization of the solution in forming the aerosol. Various conventional surfactants can be employed, such as polyoxyethylene fatty acid esters and alcohols, and polyoxyethylene sorbitan fatty acid esters. Amounts will generally range between 0.001% and 4% by weight of the formulation. An especially preferred surfactant for purposes of this invention is polyoxyethylene sorbitan monooleate.

Specific formulations and methods of generating suitable dispersions of liquid particles of the invention are described in WO 9420069, U.S. Pat. Nos. 5,915,378, 5,960,792, 5,957,124, 5,934,272, 5,915,378, 5,855,564, 5,826,570 and 5,522,385 which are hereby incorporated by reference.

Three specific examples of commercially available nebulizers suitable for the practice of this invention are the Ultravent nebulizer, manufactured by Mallinckrodt, Inc., St. Louis, Mo., the Acorn II nebulizer, manufactured by Marquest Medical Products, Englewood, Colo., and the AERx pulmonary drug delivery system manufactured by Aradigm Corporation, Hayward, Calif.

Conjugate formulations for use with a metered dose inhaler device will generally comprise a finely divided powder. This powder may be produced by lyophilizing and then milling a liquid conjugate formulation and may also contain a stabilizer such as human serum albumin (HSA). Typically, more than 0.5% (w/w) HSA is added. Additionally, one or more sugars or sugar alcohols may be added to the preparation if necessary. Examples include lactose maltose, mannitol, sorbitol, sorbitose, trehalose, xylitol, and xylose. The amount added to the formulation can range from about 0.01 to 200% (w/w), preferably from approximately 1 to 50%, of the conjugate present. Such formulations are then lyophilized and milled to the desired particle size.

The properly sized particles are then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device. An example of a commercially available metered dose inhaler suitable for use in the present invention is the Ventolin metered dose inhaler, manufactured by Glaxo Inc., Research Triangle Park, N.C.

Such conjugate formulations for powder inhalers will comprise a finely divided dry powder containing conjugate and may also include a bulking agent, such as lactose, sorbitol, sucrose, or mannitol in amounts which facilitate dispersal of the powder from the device, e.g., 50% to 90% by weight of the formulation. The particles of the powder shall have aerodynamic properties in the lung corresponding to particles with a density of about 1 g/cm² having a median diameter less than 10 micrometers, preferably between 0.5 and 5 micrometers, most preferably of between 1.5 and 3.5 micrometers.

An example of a powder inhaler suitable for use in accordance with the teachings herein is the Spinhaler powder inhaler, manufactured by Fisons Corp., Bedford, Mass.

The powders for these devices may be generated and/or delivered by methods disclosed in U.S. Pat. No. 5,997,848, 5,993,783, 5,985,248, 5,976,574, 5,922,354, 5,785,049 and 5,565,4007.

The pharmaceutical composition containing the conjugate of the invention may be administered by a wide range of mechanical devices designed for pulmonary delivery of therapeutic products, including but limited to nebulizers, metered dose inhalers, and powder inhalers, all of which are familiar to those of skill in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; the Spinhaler powder inhaler, manufactured by Fisons Corp., Bedford, Mass.; the “standing cloud” device of Inhale Therapeutic Systems, Inc., San Carlos, Calif.; the AIR inhaler manufactured by Alkermes, Cambridge, Mass.; and the AERx pulmonary drug delivery system manufactured by Aradigm Corporation, Hayward, Calif.

The pharmaceutical composition of the invention may be administered in conjunction with other therapeutic agents. These agents may be incorporated as part of the same pharmaceutical composition or may be administered separately from the polypeptide or conjugate of the invention, either concurrently or in accordance with any other acceptable treatment schedule. In addition, the polypeptide, conjugate or pharmaceutical composition of the invention may be used as an adjunct to other therapies.

Accordingly, this invention provides compositions and methods for treating most types of viral infections, cancers or tumors (e.g., breast carcinoma, non-small cell lung cancer) or tumour angiogenesis, Chrohn's disease, ulcerative colitis, Guillain-Barré syndrome, glioma, idiopathic pulmonary fibrosis, abnormal cell growth, or for immunomodulation in any suitable animal, preferably mammal, and in particular human. In particular the polypeptide, conjugate or composition of the invention may be used for the treatment of multiple sclerosis (MS), such as any of the generally recognized four types of MS (benign, relapsing remitting MS (RRMS), primary progressive MS (PPMS) and secondary progressive MS (SPMS)) and for monosymptomatic MS), hepatitis, or a herpes infection (the latter treatment optionally being combined with a treatment with IL-10).

In a further aspect the invention relates to use of a conjugate of a single chain multimeric interferon beta polypeptide, comprising:

(a) a multimeric polypeptide comprising at least two monomers linked via a peptide bond or a peptide linker, wherein at least one of said monomers is an interferon beta monomer comprising an amino acid sequence that differs from that of wildtype human interferon beta in at least one introduced glycosylation site, and

(b) at least one first non-polypeptide moiety covalently attached to the multimeric polypeptide, for the manufacture of a medicament for the treatment of viral infections, cancers, tumors, or tumour angiogenesis, Chrohn's disease, ulcerative colitis, Guillain-Barré syndrome, glioma, idiopathic pulmonary fibrosis, abnormal cell growth, or for immunomodulation in any suitable animal.

In a further aspect the invention relates to use of a conjugate of a single chain multimeric interferon beta polypeptide, comprising:

(a) a multimeric polypeptide comprising at least two monomers linked via a peptide bond or a peptide linker, wherein at least one of said monomers is an interferon beta monomer comprising an amino acid sequence that differs from that of wildtype human interferon beta in at least one introduced glycosylation site, and

(b) at least one first non-polypeptide moiety covalently attached to the multimeric polypeptide, for the manufacture of a medicament for the treatment of benign multiple sclerosis, relapsing remitting multiple sclerosis, primary progressive multiple sclerosis, secondary progressive multiple sclerosis, monosymptomatic multiple sclerosis, hepatitis, or a herpes infection, in any suitable animal.

In one embodiment said suitable animal is a mammal, such as a human.

In a further aspect the invention relates to a method of treating a mammal having circulating antibodies against interferon β 1a, such as Avonex™ or Rebif®, or 1b, such as Betaseron®, which method comprises administering a compound which has the bioactivity of interferon β and which has a reduced or no reaction with said antibodies.

In a further aspect the invention relates to a method of treating a mammal with benign multiple sclerosis, relapsing remitting multiple sclerosis, primary progressive multiple sclerosis, secondary progressive multiple sclerosis, monosymptomatic multiple sclerosis, hepatitis, a herpes infection, a viral infection, cancers, tumors, or tumour angiogenesis, Chrohn's disease, ulcerative colitis, Guillain-Barré syndrome, glioma, idiopathic pulmonary fibrosis, or abnormal cell growth, said mammal having circulating antibodies against interferon β 1a and/or 1b, which method comprises administering to said mammal a compound of the invention.

The compound is administered in an effective amount. The compound is preferably a conjugate as described herein and the mammal is preferably a human being. The mammals to be treated may suffer from any of the diseases listed above for which interferon β is a useful treatment. In particular, this aspect of the invention is of interest for the treatment of multiple sclerosis (any of the types listed above) or cancer. Furthermore, the invention relates to a method of making a pharmaceutical product for use in treatment of mammals having circulating antibodies against interferon β 1a, such as Avonex™ or Rebif®, or 1b, such as Betaseron®, wherein a compound which has the bioactivity of interferon β and which does not react with such is formulated into an injectable or otherwise suitable formulation. The term “circulating antibodies” is intended to indicate antibodies, in particular neutralizing antibodies, formed in a mammal in response to having been treated with any of the commercially available interferon β preparations (Rebif, Betaseron, Avonex).

In a further aspect the invention relates to a method of treating a patient in need of treatment with a pharmaceutical composition with at least some of the therapeutically beneficial properties of interferon β comprising administering a composition comprising a compound with at least part of the therapeutically beneficial activity of interferon β, said treatment having reduced or removed adverse psychological effects as compared to treatment with interferon β, wherein said compound is a non-naturally occurring conjugate of a polypeptide with interferon β activity and a non-polypeptide moiety, in particular a conjugate according to the present invention.

In a still further aspect the invention relates to a pharmaceutical composition for the treatment of a patient in need of treatment with a compound having at least part of the therapeutically beneficial properties of interferon β, said composition comprising a compound which is a non-naturally occurring conjugate of interferon β and a non-polypeptide moiety, said treatment further giving rise to fewer adverse psychological effects than treatment with interferon β. The conjugate is preferably a conjugate of the invention.

Also contemplated is use of a nucleotide sequence encoding a polypeptide of the invention in gene therapy applications. In particular, it may be of interest to use a nucleotide sequence encoding a polypeptide as described in the section above entitled “Glycosylated Polypeptides of the Invention modified to incorporate additional glycosylation sites.” The glycosylation of the polypeptides is thus achieved during the course of the gene therapy, i.e., after expression of the nucleotide sequence in the human body.

Gene therapy applications contemplated include treatment of those diseases in which the polypeptide is expected to provide an effective therapy due to its antiviral activity, e.g., viral diseases, including hepatitis such as hepatitis C, and particularly HPV, or other infectious diseases that are responsive to interferon β or infectious agents sensitive to interferon β. Furthermore, the conjugate or polypeptide of the invention may be used in the treatment of chronic inflammatory demyelinating polyradiculoneuropathy, and of severe necrotising cutaneous lesions. Also, gene therapy in connection with the treatment of any MS type is contemplated. Similarly, this invention contemplates gene therapy applications for immunomodulation, as well as in the treatment of those diseases in which interferon β is expected to provide an effective therapy due to its antiproliferative activity, e.g., tumors and cancers, or other conditions characterized by undesired cell proliferation, such as restenosis. A further description of such gene therapy is provided in WO 95/25170.

Local delivery of interferon β using gene therapy may provide the therapeutic agent to the target area while avoiding potential toxicity problems associated with non-specific administration.

Both in vitro and in vivo gene therapy methodologies are contemplated.

Several methods for transferring potentially therapeutic genes to defined cell populations are known. For further reference see, e.g., Mulligan, “The Basic Science Of Gene Therapy,” Science, 260, pp. 926-31 (1993). These methods include:

Direct gene transfer, e.g., as disclosed by Wolff et al., “Direct Gene transfer Into Mouse Muscle In vivo,” Science 247, pp. 1465-68 (1990);

Liposome-mediated DNA transfer, e.g., as disclosed by Caplen et al., “Liposome-mediated CFTR Gene Transfer to the Nasal Epithelium Of Patients With Cystic Fibrosis” Nature Med., 3,pp. 39-46 (1995); Crystal, “The Gene As A Drug,” Nature Med., 1,pp.- 15-17 (1995); Gao and Huang, “A Novel Cationic Liposome Reagent For Efficient Transfection of Mammalian Cells,” Biochem.Biophys Res. Comm., 179, pp. 280-85 (1991);

Retrovirus-mediated DNA transfer, e.g., as disclosed by Kay et al., “In vivo Gene Therapy of Hemophilia B: Sustained Partial Correction In Factor IX-Deficient Dogs,” Science, 262,pp. 117-19 (1993); Anderson, “Human Gene Therapy,” Science, 256,pp.808-13(1992);

DNA Virus-mediated DNA transfer. Such DNA viruses include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., All et al., “The Use Of DNA Viruses as Vectors for Gene Therapy,” Gene Therapy, 1, pp. 367-84 (1994); U.S. Pat. No. 4,797,368, and U.S. Pat. No. 5,139,941.

The invention is further described in the following examples. The examples should not, in any manner, be understood as limiting the generality of the present specification and claims.

Materials and Methods

Materials

HeLa cells—(available from American Type Culture Collection (ATCC)

ISRE—Luc (Stratagene, La Jolla USA)

pCDNA 3.1/hygro (Invitrogen, Carlsbad USA)

pGL3 basic vector (Promega)

Human genomic DNA (CloneTech, USA)

DMEM medium: Dulbecco's Modified Eagle Media (DMEM), 10% fetal bovine serum (available from Life Technologies A/S, Copenhagen, Denmark)

Assays

Interferon Assay Outline

It has previously been published that interferon β interacts with and activates Interferon type I receptors on HeLa cells. Consequently, transcription is activated at promoters containing an Interferon Stimulated Response Element (ISRE). It is thus possible to screen for agonists of interferon receptors by use of an ISRE coupled luciferase reporter gene (ISRE-luc) placed in HeLa cells.

Primary Assay

HeLa cells are co-transfected with ISRE-Luc and pCDNA 3.1/hygro and foci (cell clones) are created by selection in DMEM media containing Hygromycin B. Cell clones are screened for luciferase activity in the presence or absence of interferon β. Those clones showing the highest ratio of stimulated to unstimulated luciferase activity are used in further assays.

To screen muteins, 15,000 cells/well are seeded in 96 well culture plates and incubated overnight in DMEM media. The next day muteins as well as a known standard are added to the cells in various concentrations. The plates are incubated for 6 hours at 37° C. in a 5% CO₂ air atmosphere LucLite substrate (Packard Bioscience, Groningen The Netherlands) is subsequently added to each well. Plates are sealed and luminescence measured on a TopCount luminometer (Packard) in SPC (single photon counting) mode. Each individual plate contains wells incubated with interferon β as a stimulated control and other wells containing normal media as an unstimulated control. The ratio between stimulated and unstimulated luciferase activity serves as an internal standard for both mutein activity and experiment-to-experiment variation.

Secondary Assay

Currently, there are 18 non-allelic interferon α genes and one interferon β gene. These proteins exhibit overlapping activities and thus it is critical to ensure that muteins retain the selectivity and specificity of interferon β.

The β-R1 gene is activated by interferon β but not by other interferons. The transciption of β-R1 thus serves as a second marker of interferon β activation and is used to ensure that muteins retain interferon β activity. A 300 bp promoter fragment of β-R1 shown to drive interferon sensitive transcription (Rani. M. R. et al (1996) JBC 271 22878-22884) was isolated by PCR from human genomic DNA and inserted into the pGL3 basic vector (Promega). The resulting β-R1 luciferase gene is used in assays similar to the primary assay described above. In astrocytoma cells, the resulting β-R1 :luciferase gene has been described to show 250-fold higher sensitivity to interferon β than to interferon α (Rani et al. op cit).

ELISA Assay

The concentration of IFN-β is quantitated by use of a commercial sandwich immunoassay (PBL Biomedical Laboratories, New Brunswick, N.J., USA). The kit is based on an ELISA with monoclonal mouse anti-IFN-β antibodies for catching and detection of IFN-β in test samples. The detecting antibody is conjugated to biotin.

Tests samples and recombinant human IFN-β standard are added in 0.1 mL in concentrations from 10-0.25 ng/mL to microtiter plates, precoated with catching antibody. The plates are incubated at RT for 1 hr. Samples and standard are diluted in kit dilution buffer. The plates are washed in the kit buffer and incubated with the biotinylated detecting antibody in 0.1 mL for 1 hr at RT. After another wash the streptavidin-horseradishperoxidase conjugate is added in 0.1 mL and incubated for 1 hr at RT.

The reaction is visualised by addition of 0.1 mL Tetramethylbenzidine (TMB) substrate chromogen. The plates are incubated for 15 minutes in the dark at RT and the reaction is stopped by addition of stop solution. The absorbanse is read at 450nm using an ELISA reader.

Receptor Binding Assay

The receptor binding capability of a polypeptide or conjugate of the invention can be determined using the assay described in WO 95/25170 entitled “Analysis Of IFN-β(Phe₁₀₁) For Receptor Binding” (which is based on Daudi or A549 cells). Soluble domains of IFNAR1 and IFNAR2 can be obtained essentially as described by Arduini et al, Protein Science, 1999, vol. 8, 1867-1877 or as described in Example 9 herein.

Alternatively, the receptor binding capability is determined using a crosslinking agent such as disuccinimidyl suberate (DSS) available from Pierce, Rockford, Ill., USA as follows:

The polypeptide or conjugate is incubated with soluble IFNAR-2 receptor in the presence or absence of DSS in accordance with the manufacturer's instructions. Samples are separated by SDS-PAGE, and a western blot using anti-interferon β or anti-IFNAR2 antibodies is performed. The presence of a functional interferon β polypeptide/conjugate: receptor interaction is apparent by an increase in the molecular size of receptor and interferon β in the presence of DSS.

Furthermore, a crosslinking assay using a polypeptide or conjugate of the invention and both receptor subunits (IFNAR-1 and IFNAR-2) can establish Interferon receptor 1 binding ability. In this connection it has been published that IFNAR-l binds only after an interferon β: IFNAR-2 complex is formed (Mogensen et al., Journal of Interferon and Cytokine Research, 19:1069-1098, 1999).

In Vitro Immunogenicity Tests of Interferon β Conjugates

Reduced immunogenicity of a conjugate or polypeptide of the invention is determined by use of an ELISA method measuring the immunoreactivity of the conjugate or polypeptide relative to a reference molecule or preparation. The reference molecule or preparation is normally a recombinant human interferon β preparation such as Avonex, Rebif or Betaseron, or another recombinant human interferon β preparation produced by a method equivalent to the way these products are made. The ELISA method is based on antibodies from patients treated with one of these recombinant interferon β preparations. The immunogenicity is considered to be reduced when the conjugate or polypeptide of the invention has a statistically significant lower response in the assay than the reference molecule or preparation.

Another method of determining immunogenicity is by use of sera from patients treated with interferon beta (i.e., any commercial interferon β product) in an analogous manner to that described by Ross et al. J. Clin Invest. 95, 1974-78, 1995. In the antiviral neutralisation bioassay reduced immunogenicity results in reduced inhibition of a conjugate of the invention by patient sera compared to a wt IFN-beta reference molecule. Furthermore, in the biochemical IFN binding assay a less immunogenic conjugate is expected to bind to patient IgG to a lesser extent than reference IFN-beta molecules.

For the neutralisation assay, the reference and conjugate molecules are added in a concentration that produces approximately 80% virus protection in the antiviral neutralisation bioassay. The IFN-β proteins are mixed with patient sera in various dilutions (starting at 1:20).

Antiviral Activity

The antiviral bioassay is performed using A549 cells (CCL 185, American tissue culture collection) and Encephalomyocarditis (EMC) virus (VR-129B, American tissue culture collection).

The cells are seeded in 96 well tissue culture plates at a concentration of 10,000 cells/well and incubated at 37° C. in a 5% CO₂ air atmosphere. A polypeptide or conjugate of the invention is added in concentrations from 100-0.0001 IU/mL in a total of 100 μl DMEM medium containing fetal calf serum and antibiotics.

After 24 hours the medium is removed and 0.1 mL fresh medium containing EMC virus is added to each well. The EMC virus is added in a concentration that causes 100% cell death in IFN-β free cell cultures after 24 hours.

After another 24 hrs, the antiviral effect of the polypeptide or conjugate is measured using the WST-1 assay. 0.01 mL WST-1 (WST-1 cell proliferation agent, Roche Diagnostics GmbH, Mannheim, Germany) is added to 0.1 mL culture and incubated for ½-2 hours at 37° C. in a 5% CO₂ air atmosphere The cleavage of the tetrazolium salt WST-1 by mitochondrial dehydrogenases in viable cells results in the formation of formazan that is quantified by measuring the absorbance at 450 nm.

Neutralisation of Activity in Interferon Stimulated Response Element (ISRE) Assay

The interferon β neutralising effect of anti-interferon β sera are analysed using the ISRE-Luciferase activity assay.

Sera from interferon β treated patients or from immunised animals are used. Sera are added either in a fixed concentration (dilution 1:20-1:500 (pt sera) or 20-600 ng/mL (animal sera)) or in five-fold serial dilutions of sera starting at 1/20 (Pt sera) or 600 ng/mL (animal sera). Interferon P is added either in five fold-dilutions starting at 25.000 IU/mL or in a fixed concentration (0.1-10 IU/mL) in a total volume of 80 μl DMEM medium+10% FCS. The sera are incubated for 1 hr. at 37° C. with IFN-β.

The samples are then transferred to 96 well tissue culture plates containing HeLa cells transfected with ISRE-Luc grown from 24 hrs before (15,000 cells/well) in DMEM media. The cultures are incubated for 6 hours at 37° C. in a 5% CO₂ air atmosphere. LucLite substrate (Packard Bioscience, Groningen, The Netherlands) is subsequently added to each well. Plates are sealed and luminescence measured on a TopCount luminometer (Packard) in SPC (single photon counting) mode.

When interferon β samples are titrated in the presence of a fixed amount of serum, the neutralising effect was defined as fold inhibition (FI) quantified as EC50(w. serum)/EC50 (w/o serum). The reduction of antibody neutralisation of interferon β variant proteins is defined as

$$( 1 - \frac{FI variant}{FI wt} ) \times 100 \%$$

Biological Half-Life Measurement of a PEG-Interferon β Conjugate

Measurement of biological half-life can be carried out in a number of ways described in the literature. One method is described by Munafo et al (European Journal of Neurology 1998, vol 5 No2 p 187-193), who used an ELISA method to detect serum levels of interferon β after subcutaneous and intramuscular administration of interferon β.

The rapid decrease of interferon β serum concentrations after i.v. administration has made it important to evaluate biological responses to interferon β treatment. However it is contemplated that the conjugates of the present invention will have prolonged serum half lifes also after i.v. administration making it possible to measure by e.g., an ELISA method or by the primary screening assay.

Different pharmacodynamic markers (e.g., serum neupterin and beta2 microglobulin) have also been studied (Clin Drug Invest (1999) 18(1):27-34). These can equally well be used to evaluate prolonged biological effect. These experiments may also be carried out in suitable animal species, e.g., rats.

Assays to assess the biological effects of interferon β such as antiviral, antiproliferative and immunomodulatory effects (as described in e.g., Annals of Neurology 1995 vol 37 No 1 p 7-15) can be used together with the primary and secondary screening assays described herein to evaluate the biological efficacy of the conjugate in comparison to wild type interferon β.

Finally an animal model such as the commonly used experimental autoimmune encephalomyelitis (EAE) model can be used to establish efficacy of a conjugate or polypeptide of the invention. In the EAE model immunization with myelin or myelin derived proteins elicits a disease mimicking the majority of the inflammatory and neurologic features of multiple sclerosis in humans. EAE has been used in mice, rats, rabbits, and marmosets (Cannella et al. PNAS, 95, 10100-5, 1998, Zaprianova et al. Morfologiia, 112, 25-8, 1997, Hassouna et al. J.Urology, 130, 806-10, 1983, Genain & Hauser J. Mol. Med. 75, 187-97,1997). Other models include Theiler's murine encephalomyelitis virus (TMEV) model (Murray et al. J.Neurosci. 18, 7306-14, 1998). will be used to establish efficacy of the interferon β conjugate.

PEGylation of a Receptor-Bound Interferon β Polypeptide

In order to optimize PEGylation of an interferon β polypeptide in a manner excluding PEGylation of lysines involved in receptor recognition, the following method has been developed:

The soluble domains of IFNAR1 and IFNAR2 are obtained essentially as described in Arduini et al, Protein Science (1999), vol 8: 1867-1877.

A ternary complex consisting of an interferon β polypeptide, a soluble domain of IFNAR1 and a soluble domain of IFNAR2 in a 1:1:1 stoichiometry is formed in a PBS buffer at pH 7-9. The concentration of Interferon β polypeptide is approximately 20 ug/ml or 1 uM and the receptors are present at equimolar concentration.

M-SPA-5000 from Shearwater Polymers, Inc is added at 3 different concentration levels corresponding to 5, 20 or 100 molar excess of interferon β polypeptide. The reaction time is 30 min at RT. After the 30 min reaction period, the pH of the reaction mixture is adjusted to pH 2.0 and the reaction mixture is applied to a Vydac C 18 column and eluted with an acetonitrile gradient essentially as described (Utsumi etal, J. Biochem., vol 101, 1199-1208, (1987). Alternatively and more elegantly, an isopropanol gradient can be used.

Fractions are analyzed using the primary screening assay described herein and active PEGylated interferon-β polypeptide obtained by this method stored at −80° C. in PBS, pH 7 containing 1 mg/ml HSA.

Alternatively, to the procedure described above a soluble domain of IFNAR2 is used as the only receptor component to form a binary complex. Furthermore, IFNAR2 may be immobilized on a suitable resin (e.g., Epoxy activated Sepharose 6B) according to the manufactures instructions prior to forming the binary complex. After PEGylation, the PEGylated Interferon-β is eluted with a 0.1M Glycin, pH 2 buffer and activity measured as described after pH adjustment to neutral.

Accessible Surface Area (ASA)

The computer program Access (B. Lee and F. M. Richards, J. Mol.Biol. 55: 379-400 (1971)) version 2 (Copyright (c) 1983 Yale University) are used to compute the accessible surface area (ASA) of the individual atoms in the structure. This method typically uses a probe-size of 1.4 Å and defines the Accessible Surface Area (ASA) as the area formed by the centre of the probe. Prior to this calculation all water molecules and all hydrogen atoms are removed from the coordinate set, as are other atoms not directly related to the protein. Alternative programs are available for computing ASA, e.g., the program WhatIf G.Vriend, J. Mol. Graph. (1990) 8, 52-56, electronically available at the WWW interface on http://swift.embl-heidelberg.de/servers2/ (R.Rodriguez et.al. CABIOS (1998) 14, 523-528.) using the option Accessibility to calculate the accessible molecular surface.

Fractional ASA of Side Chain

The fractional ASA of the side chain atoms is computed by division of the sum of the ASA of the atoms in the side chain with a value representing the ASA of the side chain atoms of that residue type in an extended ALA-×-ALA tripeptide. See Hubbard, Campbell & Thornton (1991) J.Mol.Biol.220,507-530. For this example the CA atom is regarded as a part of the side chain of Glycine residues but not for the remaining residues. The following table indicates the 100% ASA standard for the side chain:

| | |
|---|---|
| Ala | 69.23 Å² |
| Arg | 200.35 Å² |
| Asn | 106.25 Å² |
| Asp | 102.06 Å² |
| Cys | 96.69 Å² |
| Gln | 140.58 Å² |
| Glu | 134.61 Å² |
| Gly | 32.28 Å² |
| His | 147.00 Å² |
| Ile | 137.91 Å² |
| Leu | 140.76 Å² |
| Lys | 162.50 Å² |
| Met | 156.08 Å² |
| Phe | 163.90 Å² |
| Pro | 119.65 Å² |
| Ser | 78.16 Å² |
| Thr | 101.67 Å² |
| Trp | 210.89 Å² |
| Tyr | 176.61 Å² |
| Val | 114.14 Å² |

Determining Surface Exposed Amino Acid Residues

The three-dimensional crystal structure of human interferon beta at 2.2 Å resolution (Karpusas etal. Proc. Nat. Acad. Sci. USA (1997) 94:11813-11818 is available from the Protein Data Bank (PDB) (Bernstein et.al. J. Mol. Biol. (1977) 112 pp. 535) and electronically available via The Research Collaboratory for Structural Bioinformatics PDB at http://www.pdb.org/ under accession code 1AU1. This crystal structure contain two independent molecules of human interferon beta in this example the A molecule is used.

Surface Exposure:

Using the WhatIf program as described above the following residues were found to have zero surface accessibility for their side chain atoms (for Gly the accessibility of the CA atom is used): G7, N14, C17, L21, I44, A55, A56, T58, I59, M62, L63, L98, L22, Y125, I129, L133, A142, W143, V146, I150, N153, I157, L160, T161, and L164.

Fractional Surface Exposure

For further analysis it was necessary to remodel the side chains of residues R7 1, R113, K115, L116, M117 due to steric clashes. The remodelling was done using Modeler 98, MSI INC. Performing fractional ASA calculations using the Access computer program on the remodelled interferon beta molecule (only including the amino acid residues and excluding the N-linked sugar moiety) resulted in the following residues having more than 25% of their side chain exposed to the surface: S2, N4, L5, F8, L9, R11, S12, F15, Q16 Q18, K19, W22, Q23, G26, R27, L28, E29, Y30, L32, K33, R35, M36, N37, D39, E42, K45, Q46, L47, Q48, Q49, Q51, K52, Q64, A68, R71, Q72, D73, S75, S76, G78, N80, E81, T82, E85, N86, A89, Y92, H93, N96, H97, K99, T100, E103, E104, K105, E107,K108, E109, D110, F111, R113, G114, K115, L116, S119, L120, H121, K123, R124, G127, R128, L130, H131, K134, A135, K136, E137, Y138, S139, H140, V148, R152, Y155, N158, G162, Y163, R165, and N166. and the following residues have more than 50% of their side chain exposed to the surface: N4, L5, F8, S12, F15, Q16, K19, W22, G26, R27, E29, Y30, K33, R35, N37, D39, E42, Q46, Q48, Q49, Q51, K52, R71, D73, S75, G78, N80, E81, T82, E85, N86, A89, Y92, H93, K99, T100, E103, E104, E107, K108, D110, F111, L116, K123, R124, G127, H131, K134, E137, V148, Y155, R165, and N166.

Nucleotide Sequence Modification Methods for Design of Peptide Addition

For example, a peptide addition may be constructed from two or more nucleotide sequences encoding the multimeric interferon β with a peptide addition, the sequences being sufficiently homologous to allow recombination between the sequences, in particular in the part thereof encoding the peptide addition. The combination of nucleotide sequences or sequence parts is conveniently conducted by methods known in the art, for instance methods which involve homologous cross-over such as disclosed in U.S. Pat. No. 5,093,257, or methods which involve gene shuffling, i.e., recombination between two or more homologous nucleotide sequences resulting in new nucleotide sequences having a number of nucleotide alterations when compared to the starting nucleotide sequences. In order for homology based nucleic acid shuffling to take place the relevant parts of the nucleotide sequences are preferably at least 50% identical, such as at least 60% identical, more preferably at least 70% identical, such as at least 80% identical. The recombination can be performed in vitro or in vivo. Examples of suitable in vitro gene shuffling methods are disclosed by Stemmer et al (1994), Proc. Natl. Acad. Sci. USA; vol. 91, pp. 10747-10751; Stemmer (1994), Nature, vol. 370, pp. 389-391; Smith (1994), Nature vol. 370, pp. 324-325; Zhao et al., Nat. Biotechnol. 1998, Mar; 16(3): 258-61;Zhao H. and Arnold, F B, Nucleic Acids Research, 1997,Vol. 25. No. 6pp. 1307-1308; Shao et al., Nucleic Acids Research 1998, Jan 15, 1998 26(2): pp. 681-83; and WO 95/17413. Example of a suitable in vivo shuffling method is disclosed in WO 97/07205.

Furthermore, a peptide addition can be constructed by preparing a randomly mutagenized library, conveniently prepared by subjecting a nucleotide sequence encoding the multimeric interferon β polypeptide with the peptide addition to random mutagenesis to create a large number of mutated nucleotide sequences. While the random mutagenesis can be entirely random, both with respect to where in the nucleotide sequence the mutagenesis occurs and with respect to the nature of mutagenesis, it is preferably conducted so as to randomly mutate only the part of the sequence that encode the peptide addition. Also, the random mutagenesis can be directed towards introducing certain types of amino acid residues, in particular amino acid residues containing an attachment group, at random into the multimeric interferon β polypeptide molecule or at random into peptide addition part thereof. Besides substitutions, random mutagenesis can also cover random introduction of insertions or deletions. Preferably, the insertions are made in reading frame, e.g., by performing multiple introduction of three nucleotides as described by Hallet et al., Nucleic Acids Res. 1997, 25(9):1866-7 and Sondek and Shrotle, Proc Natl. Acad. Sci USA 1992, 89(8):3581-5.

The random mutagenesis (either of the whole nucleotide sequence or more preferably the part thereof encoding the peptide addition) can be performed by any suitable method. For example, the random mutagenesis is performed using a suitable physical or chemical mutagenizing agent, a suitable oligonucleotide, PCR generated mutagenesis or any combination of these mutagenizing agentsand/or other methods according to state of the art technology, e.g., as disclosed in WO 97/07202.

Error prone PCR generated mutagenesis, e.g., as described by J. O. Deshler (1992), GATA 9(4): 103-106 and Leung et al., Technique (1989) Vol. 1, No. 1, pp. 11-15, is particularly useful for mutagenesis of longer peptide stretches (corresponding to nucleotide sequences containing more than 100 bp) or entire genes, and are preferably performed under conditions that increase the misincorporation of nucleotides.

Random mutagenesis based on doped or spiked oligonucleotides or by specific sequence oligonucleotides, is of particular use for mutagenesis of the part of the nucleotide sequence encoding the peptide addition.

Random mutagenesis of the part of the nucleotide sequence encoding the peptide addition can be performed using PCR generated mutagenesis, in which one or more suitable oligonucleotide primers flanking the area to be mutagenized are used. In addition, doping or spiking with oligonucleotides can be used to introduce mutations so as to remove or introduce attachment groups for the relevant non-polypeptide moiety. State of the art knowledge and computer programs (e.g., as described by Siderovski D P and Mak T W, Comput. Biol. Med. (1993) Vol. 23, No. 6, pp. 463-474 and Jensen et al. Nucleic Acids Research, 1998, Vol. 26, No. 3) can be used for calculating the most optimal nucleotide mixture for a given amino acid preference. The oligonucleotides can be incorporated into the nucleotide sequence encoding the peptide addition by any published technique using e.g., PCR, LCR or any DNA polymerase or ligase.

According to a convenient PCR method the nucleotide sequence encoding the multimeric interferon β polypeptide and in particular the peptide addition thereof is used as a template and, e.g., doped or specific oligonucleotides are used as primers. In addition, cloning primers localized outside the targetted region can be used. The resulting PCR product can either directly be cloned into an appropriate expression vector or gel purified and amplified in a second PCR reaction using the cloning primers and cloned into an appropriate expression vector.

In addition to the random mutagenesis methods described herein, it is occasionally useful to employ site specific mutagenesis techniques to modify one or more selected amino acids in the peptide addition, in particular to optimise the peptide addition with respect to the number of attachment groups.

Furthermore, random elongation mutagenesis as described by Matsuura et al, op cit can be used to construct a nucleotide sequence encoding the multimeric interferon β polypeptide having a C-terminal peptide addition. Construction of a nucleotide sequence encoding a multimeric interferon β polypeptide having an N-terminal peptide addition can be constructed in an analogous way.

Also, the methods disclosed in WO 97/04079, the contents of which are incorporated herein by reference, can be used for constructing a nucleotide sequence encoding multimeric interferon β polypeptide with an N- or C-terminal peptide addition.

The nucleotide sequence(s) or nucleotide sequence region(s) to be mutagenized is typically present on a suitable vector such as a plasmid or a bacteriophage, which as such is incubated with or otherwise exposed to the mutagenizing agent. The nucleotide sequence(s) to be mutagenized can also be present in a host cell either by being integrated into the genome of said cell or by being present on a vector harboured in the cell. Alternatively, the nucleotide sequence to be mutagenized is in isolated form. The nucleotide sequence is preferably a DNA sequence such as a cDNA, genomic DNA or synthetic DNA sequence.

Subsequent to the incubation with or exposure to the mutagenizing agent, the mutated nucleotide sequence, normally in amplified form, is expressed by culturing a suitable host cell carrying the nucleotide sequence under conditions allowing expression to take place. The host cell used for this purpose is one, which has been transformed with the mutated nucleotide sequence(s), optionally present on a vector, or one which carried the nucleotide sequence during the mutagenesis, or any kind of gene library.

Design of Peptide Addition

One example of a useful guide for designing an N-terminal peptide addition containing N-glycosylation sites is characterized by the following formula:

Y¹(NXT/S)Y²(NXT/S)_{z}Y³−P

wherein each of Y¹, Y² and Y³ independently is absent or 1, 2, 3 or 4 amino acid residues of any type, X a single amino acid residue of any type except for proline, Z any integer between 0 and 6, T/S a threonine or serine residue, preferably a threonine residue, and N and P has the meaning defined elsewhere herein.

In a first step about 10 different muteins are made that has the above formula. For instance, the about 10 muteins are designed on the basis that each of Y¹, Y² and Y³ independently is 1 or 2 alanine residues or is absent, Z any integer between 0 and 5, T/S threonine, and X alanine. Based on, e.g., in vitro bioactivity and half-life results obtained with these muteins (or any other relevant property), optimal number(s) of amino acids and glycosylation(s) can be determined and new muteins can be constructed based on this information. The process is repeated until an optimal glycosylated polypeptide is obtained. Alternatively, random mutagenesis may be used for creating N-terminally extended polypeptides. For instance, a random mutagenized library is made on the basis of the above formula. Doped oligonucleotides are synthesized coding for one amino acid residue in position X (the amino acid residue being different from proline), each of Y¹, Y² and Y³ independently is 0, 1 or 2 amino acid residues of any type, Z is 2 and T is threonine and used for constructing the random mutagenized library.

One example of a useful guide for designing an N-terminal peptide addition containing a PEGylation attachment group is characterized by the following formula using a lysine residue as an example of a PEGylation site. It will be understood that peptide additions with other attachment groups can be designed in an analogous way.

Y¹(K)Y²(K)_{z}Y³−P

wherein each of Y¹, Y² and Y³ independently is 0, 1, 2, 3 or 4 amino acid residues of any type except lysine, Z an integer between 0 and 6, K lysine, and P is as defined elsewhere herein.

In a first step about 10 different muteins are made that has the above formula. For instance, the about 10 muteins are designed on the basis that each of Y¹, Y² and Y3 independently is 1 or 2 alanine residues or is absent, Z any integer between 0 and 5, and X alanine. The muteins are then PEGylated with 10 kDa PEG (e.g., using mPEG-SPA). Based on, e.g., in vitro bioactivity and half-life results obtained with these muteins (or any other relevant property), optimal number(s) of amino acids and PEGylation sites can be determined and new muteins can be constructed based on this information. The process is repeated until an optimal PEGylated polypeptide is obtained.

Alternatively, random mutagenesis may be performed by making a random mutagenized library based on the above formula. Doped oligonucleotides are synthesized coding for one amino acid residue in position X (expect proline) and each of Y¹, Y² and Y³ independently is 0, 1 or 2 amino acid residues of any type, and Z is 2 and used for constructing the random mutagenized library.

### EXAMPLE 1

Design of an Expression Cassette for Expression of Human Wildtype Interferon β in Mammalian and Insect Cells

The DNA sequence, GenBank accession number M28622 (shown in SEQ ID NO 1), encompassing a full length cDNA encoding human interferon β with its native signal peptide, was modified in order to facilitate high expression in mammalian cells. First the ATG start codon context was modified according to the Kozak consensus sequence (Kozak, M. J. Mol Biol Aug. 20, 1987; 196(4):947-50), such that there is a perfect match to the consensus sequence upstream of the ATG start codon. Secondly codons were modified by making a bias in the codon usage towards the codons frequently used in highly expressed human genes. Subsequently, certain nucleotides in the sequence were substituted with others in order to introduce recognition sites for DNA restriction endonucleases. The gene was synthesised from 70'mer overlapping oligonucleotides by PCR using Platinum Pfx- polymerase kit (Life Technologies) resulting in the sequence shown in SEQ ID NO 3.

The synthesised gene was cloned into pcDNA3.1/Hygro (Invitrogen) between the HindIII site at the 5′ end and the BamHI at the 3′ end, resulting in pCBProF 1.

The synthetic intron from pCI-Neo (Promega) was amplified using standard PCR conditions as described above and the primers:

| | |
|---|---|
| CBProFpr37 | 5′-CCGTCAGATCCTAGGCTAGCTTATTGCGGTAGTTTATCAC-3′, |
| CBProFpr38 | 5′-GAGCTCGGTACCAAGCTTTTAAGAGCTGTAAT-3′, |

resulting in a 332 bp PCR fragment which was cut with NheI and HindIII and inserted in the 5′UTR of the plasmid pCBProF1 resulting in pCBProF4.

Codons for individual amino acids were changed by amplifying relevant regions of the coding region by PCR in such a way that the PCR introduced changes in the sequence can be introduced in the expression plasmids by classical cloning techniques. E.g., the primers:

LYS45arg-5′primer (NarI/KasI):
5′GCTGAACGGGCGCCTGGAGTACTGCCTGAAGGACAGGATGAACTTCGA
CATCCCCGAGGAAATCCGCCAGCTGCAGC-3′,
Lys45mut-3′primer (BsiWI):
5′TCTCCACGCGTACGATGGTCCAGGCGCAGTGGCTG-3′,

were used to introduce a K45R substitution in the PCR-fragment spanning the region from position 1055 to 1243 in pCBProF1. Both the PCR fragment and pCBProF1 was cut with the unique NarI and BsiWI. The PCR fragment and the vector backbone of pCBProF1 were purified and ligated resulting in substitution of the Lys45 codon AAG with the Arg codon CGC in pCBProF1.

Furthermore, SOE (sequence overhang extension) PCR was used for introduction of amino acid substitutions. In the SOE-PCR both the N-terminal part and the C-terminal part of the INFβ molecule were first amplified in individual primary PCRs.

For these primary PCRs the central complementary primers were synthesised such that the codon(s) for the amino acid(s) to be substituted is/are changed to the desired codon(s). The terminal primers were standard primers defining the N- and C-terminal of the INFβ molecule respectively. Further the terminal primers provided a restriction enzyme site enabling subsequent cloning of the full-length PCR product. Thus, the central (nonsense) primer and the N-terminal (sense) primer were used to amplify the N-terminal part of the INFβ coding region in one of the primary PCRs and equivalently for the C-terminal part. Once amplified the N- and C-terminal parts are assembled into the full-length product in a secondary PCR and cloned into a modified version of pCDNA3.1 /Hygro as described above. For instance, the following primers were used to introduce the mutations for the substitutions F111N and R113T:

CBProFprimer9(Sense):
CACCACACTGGACTAGTGGATCC TTATCAGTTGCGCAGGTAGCCGGTCAGGCGGTTG ATG
AAGTAGAAGT,
CBProFprimer231(Antisense):
CATCAGCTTGCCGGTGGTGTTGTCCTCCTTC,
CBProFprimer230(Sense):
GAAGGAGGACAACACCACCGGCAAGCTGATG,
CBProFprimer42(Antisense):
CACACTGGACTAGTAAGCTT TTATCAGTTGCGCAGGTAGC,

Furthermore, in cases where the introduced mutation(s) were sufficiently close to a unique restriction endo-nuclease site in the expression plasmid variant genes were constructed using construction procedure encompassing a single PCR step and a subsequent cloning. For instance, the substitution K19R was introduced by use of the PCR primer:

CBProFpr58:
GAGGAGTTCGAACTTCCAGTGCCAGCGCCTCCTGTGGCAGCTGAACG,
and

CBProFprimer9:

The PCR product was subsequently cloned using the restriction endo-nuclease sites BsiWI and BstBI.

### EXAMPLE 2

Construction of Interferon β Monomer With One Introduced Glycosylation Site

In order to insert an extra N-linked glycosylation site at position 111 in hINF-β, the synthetic gene (hinf-β) encoding hINF-β (described in example 1) was altered by site-directed PCR mutagenesis. Using BIO-X-ACT (Bioline, UK) and the plasmid PF050 [hinf-β)/pcDNA3.1(−)Hygro/Intron (a derivative of pcDNA3.1(−)Hygro (Invitrogen, USA) in which a chimeric intron obtained from pCI-neo (Promega, USA) had been inserted between the BamHI and NheI sites in the MCS of the vector] as template, two PCR reactions were performed with two overlapping primer-sets [CB41 (5′-TTTAAACTGGATCCAGCCACCATGACCAACAAG-3′)/CB55 (5′-CGGCCATAGT AGCGCTTCAGGTGCAGGGAGCTCATCAGCTTGCCGGTGGTGTTGTCCTCCTTC-3′) and CB42 (see above)/CB86 (5′-GAAGGAGGACAACACCACCGGCAAGCTGATGA GCTCCCTGCACCTGAAGCGCTACTATGGCC G-3′) resulting in two fragments of 446 and 184 base pairs, respectively. These two fragments were assembled in a third PCR with the flanking primers CB41 and CB42. The resulting gene was inserted into the mammalian expression vector pcDNA3.1 (−)Hygro/Intron and confirmed by DNA sequencing to have the correct base changes leading to the substitutions F111N and R113T in hINF-β (plasmid designated PF085).

### EXAMPLE 3

Construction of Interferon β Monomer With Another Introduced Glycosylation Site [Q49N+Q51T]

Analogously to what is described in Example 2 an extra N-linked glycosylation site was introduced in position 49 by means of the substitutions Q49N and Q51T. Using PF043 (hinf-β/pcDNA3.1 (Invitrogen, USA)) as template, two PCR reactions were performed with two overlapping primer-sets [PBR7 (5′-CGCGGATCCATATGACCAACAAGTGCCTG-3′)/PBR78 (5′-GGCGTCCTCCTTGGTGAAGTTCTGCAGCTG-3′) and PBR8 (5′-ATATATCCCAAGCTTTTATCAGTTGCGCAGGTAGCCGGT-3′)/PBR77 (5′-CAGCTGCAGAACTTCACCAAGGAGGACGCC-3′) resulting in two fragments of 228 and 369 base pairs, respectively. These two fragments were assembled in a third PCR with the flanking primers PBR7 and PBR8. The resulting gene was inserted into the mammalian expression vector pcDNA3.1(−)Hygro/Intron and confirmed by DNA sequencing to have the correct base changes leading to [Q49N,Q51T]hINF-β (plasmid designated PF104).

### EXAMPLE 4

Construction of Interferon β Monomrt With Two Introduced Glycosylation Sites

The additional glycosylation sites described in Examples 2 and 3 were introduced into human interferon β by means of the substitutions Q49N, Q51T, F111N, and R113T.

Using PF085 (described in Example 2) as template, two PCR reactions were performed with two overlapping primer-sets [PBR89 (5′CGCGGATCCAGCCACCATGACCAACAAGTGCCTG)/PBR78 (see above) and PBR8 (SEQ ID NO 34)/PBR77 (see above)] resulting in two fragments of 228 and 369 base pairs, respectively.

These two fragments were assembled in a third PCR with the flanking primers PBR89 and PBR8. The resulting gene was inserted into the mammalian expression vector pcDNA3.1 (−)Hygro/Intron and confirmed by sequencing to have the correct base changes leading to [Q49N, Q51T, F111N, R113T] hINF-β (plasmid designated PF123).

### EXAMPLE 5

Construction of Genes for Interferon β Single Chain Dimers

Using the nucleotide sequences constructed in Examples 1-4 single chain multimeric polypeptides constructs were made on the basis of wildtype human interferon β (WT), a variant thereof comprising the mutations Q49N+Q51T (G1), a variant comprising the mutations Q49N+Q51T+F111N+R113T (G2), a variant comprising the mutations C17S+Q49N+Q51T+D110F+F111N+R113T (INFB variant G8) and a variant comprising the mutations C17S+K19R+K33R+K45R+Q49N+Q51T+D110F+F111N+R113T (INFB variant C10). In one set of experiments the constructs were made such that the carboxy terminal of the first interferon β monomer in front with its native propeptide in front (for efficient secretion), was directly linked to the N-terminal of the second mature interferon β monomer. The following dimer constructs were made:

WT/WT, WT/G1, WT/G2, G1/WT, G1/G1, G1/G2, G2/G1 and G2/G2, the first mentioned monomer being N-terminal to the second as follows.

For the first interferon β monomer in the dimer a PCR product was generated from the relevant monomer construct (WT, G1 or G2) spanning the entire coding region including the coding region for the propeptide at the N-terminus but excluding the stop codons at the C-terminus using the primers:

CBProFpr259 (5′CAGGTTGTAGCTCATATTCCGGAGATACCCCGTCAGGCGGTTG), such that an overlap (underlined) to the second interferon monomer of the single chain dimer was created.

For the second interferon β monomer a PCR product was generated also from the relevant monomer construct (WT, G1 or G2) spanning the coding region of the mature interferon β and thus excluding the coding region for the propeptide at the N-terminus but including the stop codons at the C-terminus and part of the 3′UTR using the primers:

CBProFpr260

(5′GGTATCTCCGGAATATGAGCTACAACCTGCTCGGCTTCCTGCAGCGCAGTTCGA ATTTCCAGTGC) for creating the overlap to the first monomer of the dimer and CBProFpr20 (5′AAGAAGGCACAGTCGAGG). The DNA constructs for the full length single chain interferon β dimer were then assembled by SOE PCR and cloned into the variant version of pcDNA3.1 described above.

In a second series of experiments homo dimer molecules were made in which the first and the second interferon beta molecule is separated by a spacer or linker molecule. Three different linker molecules were used:

1. GlySerThrSerGlySerSerGlyLysSerSerGluGlyLysGly (L)

2. GlyGlyGlyGlySerGlyGlyGlyGlySerGlyGlyGlyGlySer (GL)

3. GlyGlyGlyGlySerGlyGlyGlyAsnSerThrGlyGlyGlySer (GLG)

A WT homo dimer with a peptide linker was made with a glycosylated peptide linker amino acid sequence; 4×GlySer3×GlyAsnSerThr3×GlySer (Glycosylation site in boldface). This homo dimer molecule is denominated WT/GLG/WT. The DNA construct for this molecule was made in same way as described above but using the primers:

CBProFpr102 (5′GGCACCTATTGGTCTTACTG) priming in the 5′UTR and

CBProFpr269 (5′GCGGGGGAGGCAGCGGTGGCGGGAACTCCACCGGTGGCGGGA GCATGAGCTACAACCTGCTCGGC) for the first monomer and CBProFpr270 (5′GCTCCCGCCACCGGTGGAGTTCCCGCCACCGCTGCCTCCCCCGCCATTCCGGAG ATACCCCGTCAG) and CBProFpr20(5′AAGAAGGCACAGTCGAGG) for the second monomer.

Similarly, the DNA constructs for following molecule were made; G8/GL/G8, G8/GLG/G8, C10/GL/C 10 and C10/GLG/C10.

### EXAMPLE 6

Expression of Interferon β Single Chain Dimers

The constructs disclosed in example 5 were transfected into CHO K1 cells for transient expression by use of Lipofectamine 2000 (Life Technologies # 11668-019) according to the manufactures recommendations. After typically 24 hours an estimation of the transient expression level was made. In some cases hygromycin B (Life Technologies # 10687-010) selection of a pool of stable clones was initiated after harvest of supernatant after transient expression. After achiving confluence in a T-25 tissue culture flask the medium was changed. The expression level of the stable pool was evaluated after 24 hours. The expression levels were:

TABLE
Expression of INFB single chain homo dimers.

| | Transient | |
|---|---|---|
| | expression | |
| | level | Expression level in stable pool |
| Homo dimer molecule | (IU/ml) | (IU/ml) |
| WT/WT | 440 | N.D. |
| WT/L/WT | 1573 | N.D. |
| WT/GL/WT | 2394 | N.D. |
| WT/GLG/WT | 2632 | N.D. |
| G2/G2 | 3603 | N.D. |
| G2/L/G2 | 8648 | N.D. |
| G2/GL/G2 | 10285 | N.D. |
| G2/GLG/G2 | 11714 | N.D. |
| G8/GL/G8 | 6069 | 17217 |
| G8/GLG/G8 | 1613 | 22387 |
| C10/GL/C10 | 3248 | 8138 |
| C10/GLG/C10 | 2532 | 6722 |

N.D. = Not Determined.

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this application and scope of the appended claims. For example, all the techniques and apparatus described above may be used in various combinations. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

We claim:

1. A single chain multimeric interferon β polypeptide comprising at least two monomers linked via a peptide bond or a peptide linker, wherein at least one of said monomers is an interferon β monomer comprising an amino acid sequence that differs from that of wildtype human interferon β in at least one introduced glycosylation site.

2. The multimeric polypeptide of claim 1, comprising two or more monomers with the same amino acid sequence.

3. The multimeric polypeptide of claim 1 comprising two interferon β monomers.

4. The multimeric polypeptide of claim 1 wherein said monomers independently comprises one or two introduced glycosylation site(s).

5. The multimeric polypeptide of claim 1 wherein at least one of the monomers is wildtype human interferon β, optionally C-terminally or N-terminally truncated.

6. The multimeric polypeptide of claim 1 wherein at least one of said monomers comprises the introduced glycosylation site Q49N+Q51T.

7. The multimeric polypeptide of claim 1 wherein at least one of said monomers comprises the introduced glycosylation site F111N+R113T.

8. The multimeric polypeptide of claim 1 wherein at least one of said monomers comprises a mutation independently selected from C17S, D110F, K19R, K33R, or K45R.

9. The multimeric polypeptide of claim 1 wherein the monomers are linked via a peptide linker.

10. The multimeric polypeptide of claim 9 wherein the peptide linker has from 5 to 30 amino acid residues.

11. The multimeric polypeptide of claim 9 wherein said linker comprises one or more glycosylation site(s).

12. The multimeric polypeptide of claim 9 wherein said linker comprises a lysine or cysteine as an attachment group.

13. A conjugate of a single chain multimeric interferon beta polypeptide, comprising:
(a) a multimeric polypeptide comprising at least two monomers linked via a peptide bond or a peptide linker, wherein at least one of said monomers is an interferon beta monomer comprising an amino acid sequence that differs from that of wildtype human interferon beta in at least one introduced glycosylation site, and
(b) at least one first non-polypeptide moiety covalently attached to the multimeric polypeptide.

14. A conjugate of the multimeric polypeptide of claim 1 comprising at least one first non-polypeptide moiety covalently attached to the multimeric polypeptide.

15. The conjugate of claim 13 or 14, wherein the first non-polypeptide moiety is a sugar moiety.

16. The conjugate of claim 13 or 14, wherein the first non-polypeptide moiety is a polymer molecule.

17. The conjugate of claim 16, wherein the polymer molecule is a linear or branched polyethylene glycol.

18. The conjugate of claim 16, wherein the polymer molecule has lysine or cysteine as an attachment group.

19. The conjugate of claim 13 or 14, further comprising at least one second non-polypeptide moiety.

20. The conjugate of claim 19, wherein the first non-polypeptide moiety is a polymer molecule and the second non-polypeptide moiety is a sugar moiety, or the first non-polypeptide moiety is a sugar moiety and the second non-polypeptide moiety is a polymer molecule.

21. The multimeric polypeptide conjugate of claim 13 or 14 comprising at least one improved property as compared to Avonex, Rebif or Betaseron, said property selected from the group consisting of reduced immunogenicity, increased functional in vivo half-life, and increased serum half-life.

22. A nucleotide sequence encoding a multimeric polypeptide of claim 1 or 13.

23. An expression vector comprising the nucleotide sequence of claim 22.

24. A host cell comprising a nucleotide sequence of claim 22 or an expression vector of claim 23.

25. The host cell of claim 24, which is a CHO, BHK, HEK293, or SF9 cell.

26. A method for preparing the conjugate of claim 13 or 14, wherein the multimeric polypeptide is reacted with a polymer molecule under conditions conducive for conjugation to the multimeric polypeptide to take place, and the conjugate is recovered.

27. A pharmaceutical composition comprising a multimeric polypeptide of claim 1 or the conjugate of claim 13 or 14 and a pharmaceutically acceptable diluent or carrier.

28. The composition of claim 27 for the treatment of viral infections, cancers, tumors, or tumour angiogenesis, Chrohn's disease, ulcerative colitis, Guillain-Barré syndrome, glioma, idiopathic pulmonary fibrosis, abnormal cell growth, or for immunomodulation in a suitable animal.

29. The composition of claim 28 for the treatment of multiple sclerosis, hepatitis, or a herpes infection.

30. A method of treating a mammal with a viral infection, cancers, tumors, or tumour angiogenesis, Chrohn's disease, ulcerative colitis, Guillain-Barré syndrome, glioma, idiopathic pulmonary fibrosis, abnormal cell growth, which method comprises administering an effective amount of the composition of claim 27.

31. A method of treating a mammal with benign multiple sclerosis, relapsing remitting multiple sclerosis, primary progressive multiple sclerosis, secondary progressive multiple sclerosis, monosymptomatic multiple sclerosis, hepatitis, or a herpes infection, which method comprises administering an effective amount of the composition of claim 27.

32. A method of treating a mammal with a viral infection, cancers, tumors, or tumour angiogenesis, Chrohn's disease, ulcerative colitis, Guillain-Barré syndrome, glioma, idiopathic pulmonary fibrosis, abnormal cell growth, benign multiple sclerosis, relapsing remitting multiple sclerosis, primary progressive multiple sclerosis, secondary progressive multiple sclerosis, monosymptomatic multiple sclerosis, hepatitis, or a herpes infection, said mammal having circulating antibodies against interferon β 1a and/or 1b, which method comprises administering an effective amount of the composition of claim 27.
